(19) United States
(12) Patent Application Publication
(10) Pub. No.: US 2024/0167172 A1
(43) Pub. Date: May 23, 2024

(54) Reactors and Methods to Reduce Carbon Footprint of Electric Arc Furnaces While Producing Sustainable Chemicals

(71) Applicant: Dioxycle, Bordeaux (FR)

(72) Inventors: Sarah Lamaison, Paris (FR); Jonathan Maistrello, La Garenne Colombes (FR); Ruperto G Mariano, Ithaca, NY (US); Bastien Faure, Paris (FR); David Wakerley, Paris (FR); Lindsay Leveen, Sausalito, CA (US)

(21) Appl. No.: 18/138,116

(22) Filed: Apr. 23, 2023

Related U.S. Application Data
(60) Provisional application No. 63/447,346, filed on Feb. 21, 2023.
(60) Provisional application No. 63/427,800, filed on Nov. 23, 2022.

(51) Int. Cl. C25B 3/25; C01B 32/942; C07C 1/32; C21B 13/12

(52) U.S. Cl. CPC C25B 3/25; C01B 32/942; C07C 1/325; C21B 13/12; C01B 2210/0006; C21B 2100/40; C21B 2100/80

(57) ABSTRACT

Methods and systems for the valorization of carbon monoxide emissions from electric arc furnaces into highly valuable low-carbon footprint chemicals using carbon monoxide electrolysis are disclosed herein are disclosed. A disclosed method includes operating an electric arc furnace, generating, via operation of the electric arc furnace, a volume of carbon monoxide, supplying the volume of carbon monoxide to a cathode area of a carbon monoxide electrolyzer to be used as a reduction substrate, and generating, using the carbon monoxide electrolyzer, the reduction substrate, and an oxidation substrate, a volume of generated chemicals. The volume of generated chemicals is at least one of: a volume of hydrocarbons, a volume of organic acids, a volume of alcohol, a volume of olefins and a volume of N-rich organic compounds.

### CROSS REFERENCE TO RELATED APPLICATIONS

This application claims the benefit of U.S. Provisional Application No. 63/447,346, filed on Feb. 21, 2023, and U.S. Provisional Application No. 63/427,800, filed Nov. 23, 2022, both of which are incorporated by reference herein in their entireties for all purposes.

### BACKGROUND

There is an urgent need to develop technologies which make the capture or valorization of carbon dioxide more economical in highly emitting sectors such as metallurgy and chemical processes. Furthermore, there is an urgent need to reduce emissions related to the production of useful fuels and chemicals in our society and to find alternative ways to produce such fuels sustainably instead of relying on fossil resource extraction and processing for their production. Accordingly, technologies that both generate useful fuels and chemicals, while at the same time using oxocarbon feedstocks that would otherwise have been emitted into the atmosphere, are critically important because they both generate useful chemicals without additional emissions and because the economic value of the useful chemicals can offset the cost of oxocarbon capture and conversion.

Electric arc furnace technologies are used in various industries that have high oxocarbon emission rates such as in metal smelting and chemical production. Historically developed for smelting iron, electric arc furnaces have also proven useful to produce different compounds such as phosphorus and calcium carbide. Since the first lab discovery and implementations of the process in the late nineteenth and early twentieth centuries, electric arc furnace technologies have now been developed at large scale in various industrial processes including ferroalloy smelting, steel production, carbon steel production, stainless or alloy steel production, non-ferrous metal melting, iron foundries, and in the chemical production industry.

Multiple types of electric arc furnaces exist including direct electric arc furnaces, indirect electric arc furnaces, and submerged electric arc furnaces. Three main kinds of electric arc furnaces are close, semi-open, and open electric arc furnaces. Open electric arc furnaces directly put into contact the solid burden or charge with air. In open furnaces, the produced carbon monoxide is oxidized into carbon dioxide. In closed furnaces, the produced gas mixture is collected and usually flared or valorized as fuel gas.

The operation of an electric arc furnace is accompanied with the release of carbon monoxide that results from the partial oxidation of solid carbon injected within the processes to reduce materials fed into the furnace, and to enforce favorable physical and environmental properties for the reactor system. Such carbon monoxide emissions may be vented directly ‘as is’ or may be fully oxidized to carbon dioxide either directly in the furnace in the case of an open reactor, fed with an air stream, or outside the reactor through flaring or after its combustion in a power generator. Although providing higher safety as they minimize the downstream handling of toxic carbon monoxide that is instead directly oxidized in the furnace, open air furnaces are less popular than semi-closed or closed furnaces as the volume of gas to be handled in the former case leads to sometimes prohibitive costs. In semi-closed or closed furnaces, there is either no combustion or limited combustion of the carbon monoxide in the furnace and most of the carbon monoxide is recovered through a separation unit. As a result, the off-gas composition of most electric arc furnaces comprises between 70% and 90% carbon monoxide in volume in the case of semi-closed and closed furnaces respectively.

In the steel industry, electric arc furnaces have been largely developed in the context of the mini-mill developments to produce secondary steel from steel scrap in opposition to the traditional fully integrated blast-furnace and basic oxygen furnace technology used to produce primary steel from iron ore. Electric arc furnaces are now being deployed more widely to produce multiple types of steel: secondary steel and primary steel, which would require the need to combine with technologies allowing for the production of iron from iron ore. Although this step of producing iron from iron ore can be carried out by a traditional blast furnace, electric arc furnaces are preferable when coupled with direct-reduced-iron-producing technologies as the heat generated during the production of direct reduced iron can be advantageously used in the electric arc furnace and the solid direct reduced iron presents similar physical properties as the steel scrap usually employed in the electric arc furnaces.

The production of steel in an electric arc furnace is accompanied by the generation of a slag. The formation of the slag also produces carbon monoxide. The slag exhibits a lower density compared to the iron and steel. Therefore, the slag floats on the top of the melting iron and advantageously provides some thermal insulation of the molten steel to reduce heat losses while protecting the furnace roof and walls from the radiant heat of the reactor. This foaming slag is usually generated by the reaction of oxygen injected into the molten steel bath with contaminants and added carbon, such as coal or coke that are supplemented to transform any iron oxide to metallic iron, leading to the co-generation of carbon monoxide.

In the chemical industry, electric arc furnaces are used to form chemicals such as calcium carbide (CaC₂). Calcium carbide is critical in the production of acetylene, a flammable gas at ambient conditions, the combustion of which is used for soldering or lighting purposes. Acetylene is also used as a precursor for vinyl chloride monomers to produce polyvinyl chloride (PVC), a highly versatile material used in many construction applications such as water, drainage and sewage pipes. The production of calcium carbide proceeds through three main steps. The first step is a limestone calcination step usually carried out in a lime kiln to produce lime:

CaCO₃(s)↔CaO(s)+CO₂(g)ΔHᵣ=168 kJ/mol (1)

Reaction (1) is highly endothermic and requires the transfer of a high amount of heat. This energy can be produced by burning fuels such as natural gas or carbon monoxide. The targeted CaO purity for this reaction is usually higher than 90%. The second step is the production of a dried carbon source. This carbon source can come from, for example, petroleum coke, metallurgical coke, or anthracite/coal coke. A dryer, requiring a heat input, is then implemented on that carbon source. The third step is the calcium carbide production step which is conducted in an electric arc furnace once the dried coke and lime have been conveyed to the electric arc furnace. The calcium carbide production step occurs according to the following equation:

CaO(s)+3 C(s)→CaC₂(s)+CO(g)ΔHᵣ=465 kJ/mol (2)

The electric arc furnace employed for the third step emits large of amounts of carbon monoxide. At least one molecule of carbon monoxide is released for every molecule of calcium carbide produced. In some cases, an excess of coal feedstock may be used to ensure that other reducible components in the lime feedstock do not diminish the efficiency of the lime production process, leading to a higher amount of carbon monoxide in the gas stream departing the electric arc furnace.

Electric arc furnace technologies applied in applications described above lead to the generation of large amount of carbon monoxide in the off-gas alongside with the main gas components such as hydrogen, methane, nitrogen and water, gas impurities such as carbon dioxide, phosphine (PH₃), sulfur oxides (SOₓ) and nitrogen oxides (NOₓ), and solid contaminants such as dust, ashes and particulate matter (PM). Due to their composition, such electric arc furnace off-gases are highly explosive, and their handling incurs significant costs. To dispose of electric arc furnace off-gases, particulates are removed from the gas stream prior to flaring the gas stream to produce carbon dioxide vented into the atmosphere. The recent increase in fossil resource costs and deployment of regulations to prevent environmental damages and health hazards associated with such venting have led the electric arc furnace users to develop solutions to capture and utilize the carbon monoxide for its heat content.

Effectively valorizing the electric arc furnace off-gas as heat is challenging because of the low calorific value of carbon monoxide. Because the gross heating value of carbon monoxide is 12,035 kJ/Nm³ (compared to that of methane, 37,669 kJ/Nm³ of methane), the use of carbon monoxide for heating purposes is sub-optimal in the context of reducing greenhouse gas emissions. For each molecule of carbon monoxide combusted or of methane combusted, one molecule of carbon dioxide will be released into the atmosphere. However, the heat generated by the combustion of one mole of carbon monoxide will be more than three-fold lower than the one generated by the combustion of one mole of methane. Furthermore, for the same amount of heat generated by burning carbon monoxide or methane, the combustion volume chamber must be much larger in the case of carbon monoxide combustion, thus leading to higher investment costs. As regulations increasingly penalize carbon dioxide emissions, such inefficiency will lead to increased operating costs and reduced profitability hence limiting the attractivity of electric arc furnace-based industrial processes.

### SUMMARY

Methods and systems for the valorization of carbon monoxide emissions from electric arc furnaces into highly valuable low-carbon footprint chemicals using carbon monoxide electrolysis are disclosed herein.

As used herein, valorization of carbon monoxide refers to the transformation of the carbon and oxygen components of carbon monoxide into more economically valuable chemicals such as hydrocarbons, organic acids, alcohols, olefins and N-rich organic compounds. Using the approaches disclosed herein, such chemicals can be produced cost-competitively with conventional petrochemical routes and contribute to the development of a circular carbon economy.

As described above, current electric arc furnace applications involving metallurgy and chemical production often lead to the production of large volumes of carbon monoxide which creates the described collection of attendant issues. Using carbon monoxide as a source of heat is generally not efficient. Furthermore, the valorization of carbon monoxide is difficult due to the nature of electric arc furnace operation. In most cases, electric arc furnaces are operated through alternate phases including the loading of the furnace, start-up of the arc, melting, stopping of the arc, and removal of the slag and molten product from the reactor (i.e., deslagging). These alternate phases hamper the valorization of the electric arc furnace off-gases through a continuous process because the carbon monoxide is only present in the off-gas from the electric arc reactor in some of these phases. The intermittent nature of carbon monoxide production also makes usage of the carbon monoxide as heat for attendant processes even less beneficial as common downstream industrial applications require the continuous provisioning of heat. These issues impose a limit on the range and number of end-users who could benefit from integration with an electric arc furnace plant.

There is a need to develop paths to efficiently handle and valorize electric arc furnace off-gases to minimize the associated carbon emissions and costs. In addition, such electric arc furnace handling and valorization paths would benefit from being able to cope with the intermittency of the electric arc furnace and generation of its off-gas.

In specific embodiments of the inventions disclosed herein, the off-gas from an electric arc furnace is not vented or used for heat but is instead processed using a gas separation system to produce purified carbon monoxide streams. In such cases, a separation unit is fluidly connected to the furnace and is fed with a volume of off-gas and produces a volume of carbon monoxide with the desired purity. The volume of carbon monoxide can then be provided to the cathode of a novel reactor, a carbon monoxide electrolyzer, where it can be used as the reduction substrate for a reduction reaction paired with an oxidation reaction at the anode of the carbon monoxide electrolyzer. One or more additive chemicals can be provided with the volume of carbon monoxide to the electrolyzer. The reduction of the volume of carbon monoxide can valorize the carbon monoxide to produce useful chemicals that would otherwise have been produced via carbon-intensive petrochemical processing. The characteristics of the produced chemicals depend on the characteristics of the electrolyzer and the additive chemicals.

In specific embodiments of the inventions disclosed herein, methods and systems are disclosed to cope with the intermittent nature of the operation of an electric arc furnace. These methods capitalize on the fast start-up time and flexibility of a carbon monoxide electrolyzer operated at low temperatures as compared to alternative approaches that could be used to valorize the carbon monoxide from the electric arc furnace.

In specific embodiments of the inventions disclosed herein, the carbon monoxide electrolyzer and electric arc furnace are operated asynchronously or at opposite times to assist with electrical power management and stabilization, and to capitalize on the intermittent nature of the electric arc furnace operation. In these embodiments, the carbon monoxide produced by the electric arc furnace can be stored temporarily in one or more storage systems that are downstream of the electric arc furnace and upstream of the carbon monoxide electrolyzer. The storage and any accompanying systems for compression can be used to either minimize downtime of the carbon monoxide electrolyzer, in which case the storage is acting as a buffer between the intermittent electric arc finance process and continuous carbon monoxide electrolyzer, or allow for the carbon monoxide electrolyzer to be used asynchronously with the electric arc furnace in order to smooth the electrical consumption of the overall system and help with electrical power management and stabilization.

In specific embodiments of the inventions disclosed herein, separation units are disclosed for the efficient recovery of carbon monoxide from electric arc furnace off-gases and its use within the carbon monoxide electrolyzer.

In specific embodiments of the inventions disclosed herein, separation constraints for the off-gas of the electric arc furnace are relaxed to decrease the capital and operational costs of the overall system. In specific applications, an electric arc furnace off-gas contains dihydrogen (H₂) gas. Instead of separating the dihydrogen from the off-gas, a separation unit can be designed to not fully remove it from the carbon-monoxide-rich stream before the latter enters the carbon monoxide electrolyzer. This limits the separation costs by leveraging the fact that small amounts of dihydrogen don't significantly affect the carbon monoxide electrolyzer.

In specific embodiments of the inventions disclosed herein, the carbon monoxide electrolyzer will produce purified oxygen in the process of valorizing the carbon monoxide from the electric arc furnace (e.g., when the anodic substrate is water). In these embodiments, the oxygen produced by the carbon monoxide electrolyzer can be cycled back to the electric arc furnace to be used in the melting or deslagging phases of operation. Electric arc furnace cycle operation includes the use of pure or enriched oxygen for the melting phase through heaters, such as oxy-fuel burners, and for the deslagging phase. Often, this oxygen is produced by PSA (Pressure Swing Adsorption) units operating directly from the air. These embodiments reduce or avoid the need for PSA units to produce oxygen for the electric arc furnace. Moreover, the purity of oxygen produced by the carbon monoxide electrolyzer would likely be higher than the purity of oxygen produced via PSA, meaning that the carbon monoxide concentration in the electric arc furnace exhaust would also be higher. In specific embodiments, a volume of oxygen gas is supplied to the electric arc furnace with an oxygen purity higher than an output of a standard pressure swing adsorption unit operating on standard ambient air. Furthermore, the purified oxygen will decrease the nitrogen content of the off-gas, reducing nitrogen oxide and nitrogen content in the furnace off-gas, thereby making the separation of the carbon monoxide from the off-gas easier to manage and more efficient.

In specific embodiments of the inventions disclosed herein, heat from the off-gas can be used to provide energy to the downstream gas separators described above, for heating the oxygen that is provided back to the electric arc furnace described above, for additional carbon dioxide capture purposes, or for the downstream processing of the carbon-monoxide-derived chemicals.

In specific embodiments of the invention disclosed herein, the oxidation substrate for the anode region of the carbon monoxide electrolyzer is dihydrogen and dihydrogen present in the off-gas from the electric arc furnace is separated and used for this purpose. In the alternative or in combination, the cathode of the electrolyzer can conduct a parasitic reduction reaction (in addition to the targeted carbon monoxide reduction reaction) which produces dihydrogen, and this dihydrogen can be provided to the anode of the carbon monoxide electrolyzer.

In specific embodiments of the inventions disclosed herein, dihydrogen separated from the off-gas of the electric arc furnace or from the carbon monoxide electrolyzer cathodic parasitic reaction is used for its heat capacity in the electric arc furnace process such as by being used to pre-heat the electric arc furnace.

In specific embodiments of the inventions disclosed herein, dihydrogen (separated from the off-gas of the electric arc furnace or from the carbon monoxide electrolyzer cathodic parasitic reaction) is valorized for hydrogenation reactions involving the chemicals produced by the electrolyzer.

In specific embodiments of the inventions disclosed herein, dihydrogen (separated from the off-gas of the electric arc furnace or from the carbon monoxide electrolyzer) can be used in a direct reduced iron (DRI) furnace to produce direct reduced iron. This direct reduced iron can be used in the electric arc furnace to generate steel.

The approaches described above decrease the cost and carbon footprint of the overall system as the costly dihydrogen feedstock is used more efficiently as a chemical reactant or replaces a carbon-based combustible.

As used herein, the term “fluid” will be used to refer to describe a substance that is in any fluidic physical form including in liquid, gaseous, supercritical or a combination of liquid and gaseous form.

In specific embodiments of the inventions disclosed herein, a method is provided. The method includes operating an electric arc furnace, generating, via operation of the electric arc furnace, a volume of carbon monoxide, supplying the volume of carbon monoxide to a cathode area of a carbon monoxide electrolyzer to be used as a reduction substrate, and generating, using the carbon monoxide electrolyzer, the reduction substrate, and an oxidation substrate, a volume of generated chemicals. The volume of generated chemicals is at least one of: a volume of hydrocarbons, a volume of organic acids, a volume of alcohol, a volume of olefins and a volume of N-rich organic compounds.

In specific embodiments of the inventions disclosed herein, a system is provided. The system comprises: an electric arc furnace; an off-gas port of the electric arc furnace for an off-gas including a volume of carbon monoxide; a carbon monoxide electrolyzer having an anode area and a cathode area; and at least one fluid connection. The volume of carbon monoxide is routed from the off-gas port to the cathode area using the at least one fluid connection.

In specific embodiments of the inventions disclosed herein, a method is provided. The method comprises: operating at least one furnace selected from a group consisting of: a direct reduced iron furnace, a blast oxygen furnace, and an electric arc furnace; generating, via operation of the at least one furnace, a volume of carbon monoxide; supplying the volume of carbon monoxide to a cathode area of a carbon monoxide electrolyzer to be used as a reduction substrate; and generating, using the carbon monoxide electrolyzer, the reduction substrate, and an oxidation substrate, a volume of generated chemicals. The volume of generated chemicals is at least one of: a volume of hydrocarbons, a volume of organic acids, a volume of alcohol, a volume of olefins and a volume of N-rich organic compounds.

### BRIEF DESCRIPTION OF THE DRAWINGS

The accompanying drawings illustrate various embodiments of systems, methods, and embodiments of various other aspects of the disclosure. A person with ordinary skills in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 illustrates a system for valorizing a volume of carbon monoxide of an off-gas stream from an electric arc furnace using a carbon monoxide electrolyzer in accordance with specific embodiments of the inventions disclosed herein.

FIG. 2 illustrates a system for valorizing a volume of carbon monoxide of an off-gas stream from an electric arc furnace using a carbon monoxide electrolyzer where the heat from the off-gas from the electric arc furnace is used by a heat recovery unit in accordance with specific embodiments of the inventions disclosed herein.

FIG. 3 illustrates a system for valorizing a volume of carbon monoxide of an off-gas stream from an electric arc furnace using a carbon monoxide electrolyzer where heat from the off-gas as recovered by a heat recovery unit is used to supply a carbon dioxide polishing unit in accordance with specific embodiments of the inventions disclosed herein.

FIG. 4 illustrates a system for valorizing a volume of carbon monoxide of an off-gas stream from an electric arc furnace using a carbon monoxide electrolyzer where heat from the off-gas as recovered by a heat recovery unit is used to supply a distillation unit for an acetic acid or acetate-containing solution in accordance with specific embodiments of the inventions disclosed herein.

FIG. 5 illustrates a system for valorizing a volume of carbon monoxide of an off-gas stream from an electric arc furnace using a carbon monoxide electrolyzer where heat from the off-gas as recovered by a heat recovery unit is used to supply a carbon dioxide recovery reactor in accordance with specific embodiments of the inventions disclosed herein.

FIG. 6 illustrates a system for valorizing a volume of carbon monoxide of an off-gas stream from an electric arc furnace using a carbon monoxide electrolyzer where heat from the off-gas as recovered by a heat recovery unit is used to supply a reverse water gas shift reactor for the conversion of carbon dioxide to carbon monoxide in accordance with specific embodiments of the inventions disclosed herein.

FIG. 7 illustrates a system for valorizing a volume of carbon monoxide of an off-gas stream from an electric arc furnace using a carbon monoxide electrolyzer with a storage unit in accordance with specific embodiments of the inventions disclosed herein.

FIG. 8 illustrates a system using an electric arc furnace and a water electrolyzer in accordance with specific embodiments of the inventions disclosed herein.

FIG. 9 illustrates a system using an electric arc furnace and a water electrolyzer in which water from a heat recovery unit operating on the off-gas of the electric arc furnace produces water that is used as an input for the water electrolyzer.

FIG. 10 illustrates a system for valorizing a volume of carbon monoxide of an off-gas stream from an electric arc furnace using a carbon monoxide electrolyzer with a non-oxidative gas separation unit used to treat the off-gas before it is provided to the carbon monoxide electrolyzer.

FIG. 11 illustrates a system for valorizing a volume of carbon monoxide of an off-gas stream from an electric arc furnace using a carbon monoxide electrolyzer with a non-oxidative gas separation unit used to treat the off-gas before it is provided to the carbon monoxide electrolyzer and illustrates how the flaring or valorization of carbon monoxide for heat and the concomitant emission of carbon dioxide can be eliminated.

FIG. 12 illustrates a block diagram of a system including a carbon monoxide electrolyzer integrated with a broader calcium carbide to acetylene process chain.

FIG. 13 illustrates a block diagram of a system including a carbon monoxide electrolyzer integrated with a broader calcium carbide to acetylene process chain in which calcium hydroxide wastes from the calcium carbonate hydrolysis are used to produce an electrolyte for operation of the carbon monoxide electrolyzer.

FIG. 14 illustrates an electrolyzer which can be used for the electrolysis of carbon monoxide in the form of a stack in accordance with specific embodiments of the invention disclosed herein.

FIG. 15 illustrates a set of example reactions in carbon monoxide electrolyzers using ion exchange membranes in accordance with specific embodiments of the invention disclosed herein.

FIG. 16 illustrates examples of reactions in carbon monoxide electrolyzers using separators in accordance with specific embodiments of the invention disclosed herein.

### DETAILED DESCRIPTION

Reference will now be made in detail to implementations and embodiments of various aspects and variations of systems and methods described herein. Although several exemplary variations of the systems and methods are described herein, other variations of the systems and methods may include aspects of the systems and methods described herein combined in any suitable manner having combinations of all or some of the aspects described.

Methods and systems for the valorization of carbon monoxide emissions from electric arc furnaces into highly valuable low-carbon footprint chemicals using carbon monoxide electrolysis in accordance with the summary above are disclosed in detail herein. The methods and systems disclosed in this section are nonlimiting embodiments of the invention, are provided for explanatory purposes only, and should not be used to constrict the full scope of the invention. It is to be understood that the disclosed embodiments may or may not overlap with each other. Thus, part of one embodiment, or specific embodiments thereof, may or may not fall within the ambit of another, or specific embodiments thereof, and vice versa. Different embodiments from different aspects may be combined or practiced separately. Many different combinations and sub-combinations of the representative embodiments shown within the broad framework of this invention, that may be apparent to those skilled in the art but not explicitly shown or described, should not be construed as precluded.

In specific embodiments of the inventions disclosed herein, carbon monoxide emissions from the output of a carbon monoxide electrolyzer are not flared or vented and are instead fed to the cathode area of an emerging kind of electrolyzer, a carbon monoxide electrolyzer, that is specifically tuned to produce highly valuable chemicals from the reduction of the carbon monoxide at the cathode area while an oxidation of an oxidation substrate is carried out at the anode of the electrolyzer. Carbon monoxide, although a waste product accounting for carbon emission and having adverse environmental impact if emitted into the atmosphere directly or further oxidized to carbon dioxide, carbon monoxide is an energy-dense substrate. Accordingly, if carbon monoxide is used as the reduction substrate of an electrolyser, the energy demand of the electrolysis can be reduced. As such, coupling a carbon monoxide electrolyzer with an electric arc furnace off-gas stream provides a route to produce highly valuable chemicals with reduced energy costs—in particular, compared to the case where the electrolysis would be performed on carbon dioxide—while also reducing the carbon oxide emission footprint and cost of the electric arc furnace.

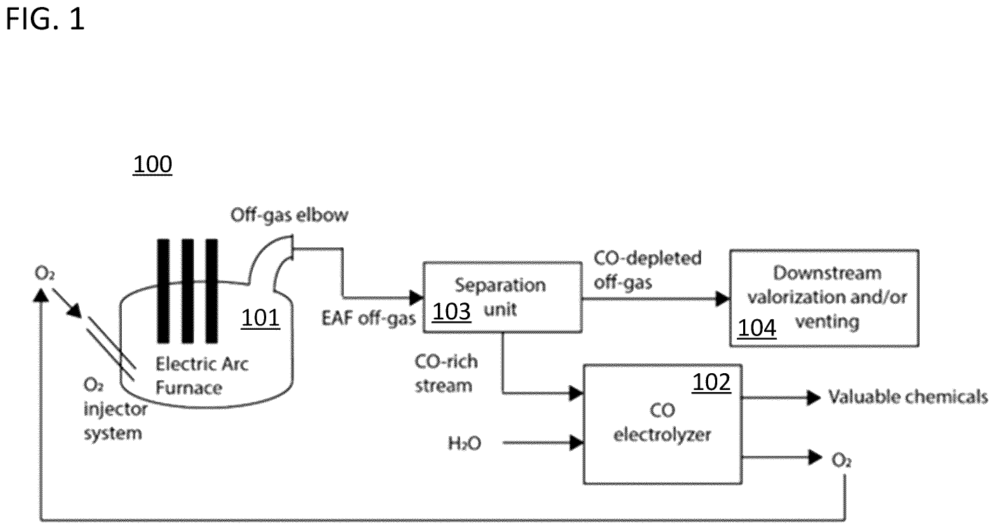

FIG. 1 illustrates a system 100 for valorizing a volume of carbon monoxide of an off-gas stream from an electric arc furnace 101 using a carbon monoxide electrolyzer 102. As illustrated, carbon monoxide electrolyzer 102 is fluidly connected to electric arc furnace 101 via a separation unit 103. The separation unit 103 provides a volume of carbon monoxide in a carbon rich stream to a cathode area of carbon monoxide electrolyzer 102 and provides a carbon monoxide depleted off-gas to a downstream valorization and or venting system 104. The separation unit 103 receives the off-gas stream from electric arc furnace 101 via an off-gas elbow of electric arc furnace 101. In the illustrated example, the oxidation substrate is water which is supplied to an anode area of carbon monoxide electrolyzer 102. The carbon monoxide electrolyzer 102 then produces valuable chemicals and purified oxygen gas. As illustrated, the purified oxygen gas can be recycled to the electric arc furnace 101 as a volume of oxygen gas. The purified oxygen gas can be injected using an oxygen injector system to be used to alter the characteristics of the material in the furnace (e.g., to oxidize any impurities from the melting material in the formation of slag), for the formation of a foaming slag (e.g., through gasification of the carbon substrate), or to power oxyfuel burners to chemically heat the electric arc furnace. The formation of the slag can be conducted using the recycled oxygen mixed with additional oxygen from another source. As illustrated, the volume of oxygen is fed to electric arc furnace 101 through dedicated oxygen injectors, but the volume of oxygen can in the alternative or in combination be provided to oxyfuel burners to chemically heat the furnace. The use of purified oxygen, instead of enriched oxygen from a PSA process, will significantly lower the nitrogen content of the electric arc furnace off-gas making the carbon monoxide purer and easier to be valorized in the carbon monoxide electrolyzer.

In specific embodiments of the inventions disclosed herein, heat from the electric arc furnace can be harvested to improve the performance of the overall system. For example, the electric arc furnace 101 could be equipped with heat recovery systems to preheat the electric arc furnace after the furnace has been cooled through one of its operational phases. As another example, heat can be recovered from a cooling system located around the off-gas uptakes that cools the electric arc-furnace off-gas from temperatures over 1000° C. to temperatures below 250° C. As another example, heat can be recovered from the electric arc furnace off-gas. The recovered heat can be used either in downstream separation processes for the purification of the carbon monoxide-derived valuable chemicals, for heating oxygen or hydrogen supplied to the electric arc furnace from the carbon monoxide electrolyzer, for powering alternative methods of carbon dioxide capture, or for other uses such as powering a carbon dioxide polishing unit upstream of, and connected to, the carbon monoxide electrolyzer.

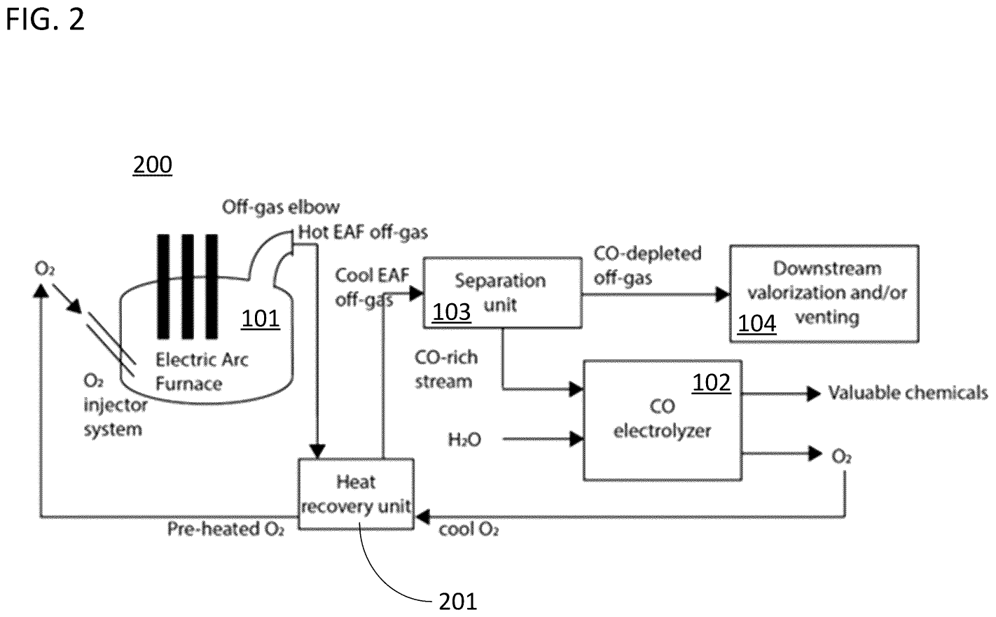

FIG. 2 illustrates a system 200 for valorizing a volume of carbon monoxide of an off-gas stream from an electric arc furnace 101 using a carbon monoxide electrolyzer 102 where the heat from the off-gas from the electric arc furnace is used by a heat recovery unit 201. The carbon monoxide electrolyzer 102 is fluidly connected to the electric arc furnace via heat recovery unit 201 and separation unit 103. As illustrated, the heat recovery unit 201 takes in a volume of cool purified oxygen from carbon monoxide electrolyzer 102 and hot off-gas from the electric arc furnace 101 and converts them into a cool stream of off-gas and pre-heated oxygen for use by the electric arc furnace 101. Heat can be recovered from the electric arc furnace off-gas to pre-heat the oxygen before it is used in the oxygen injectors or in an oxyfuel burner to maximize the heat efficiency of the reactor.

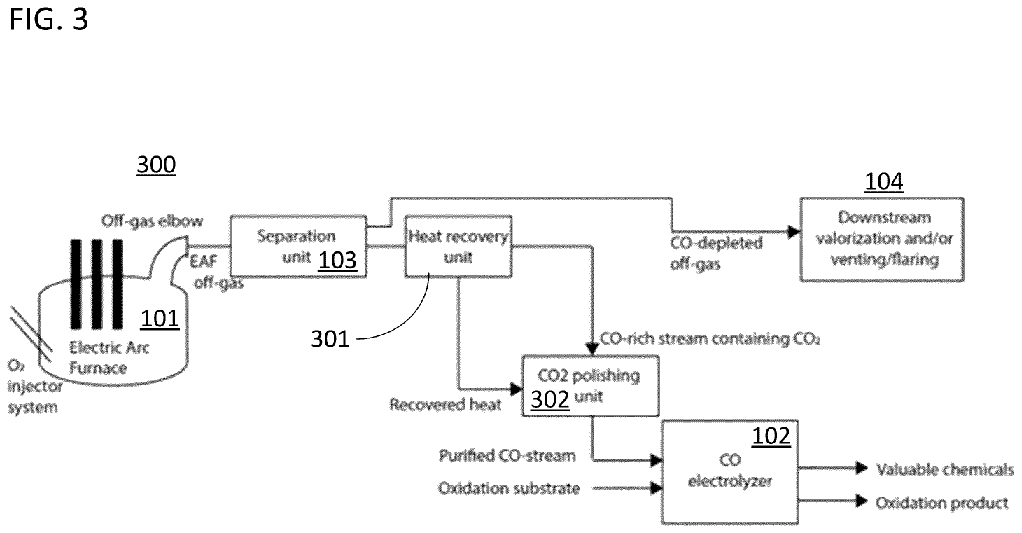

FIG. 3 illustrates a system 300 for valorizing a volume of carbon monoxide of an off-gas stream from an electric arc furnace 101 using a carbon monoxide electrolyzer 102 where heat from the off-gas as recovered by a heat recovery unit 301 is used to supply a carbon dioxide polishing unit 302. The approach is beneficial in that the acidic nature of the carbon monoxide rich stream, which contains carbon dioxide, can damage the cathode area of the carbon monoxide electrolyzer and have a negative impact on the alkaline environment of the reactor, thereby decreasing its efficiency.

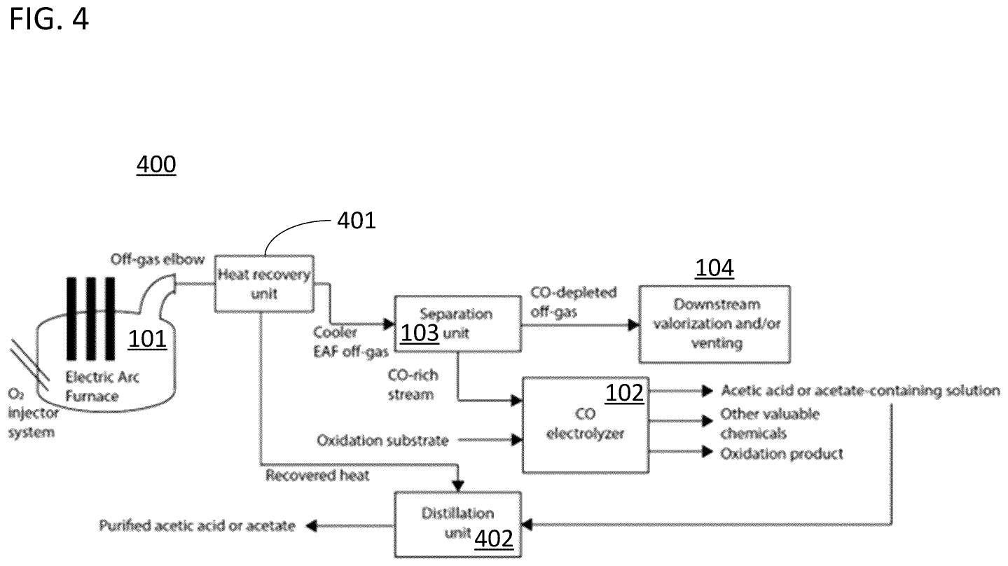

FIG. 4 illustrates a system 400 for valorizing a volume of carbon monoxide of an off-gas stream from an electric arc furnace 101 using a carbon monoxide electrolyzer 102 where heat from the off-gas as recovered by a heat recovery unit 401 is used to supply a distillation unit 402 for an acetic acid or acetate-containing solution. In the illustrated example, at least one of the valuable chemicals produced by the carbon monoxide electrolyzer 102 via the valorization of carbon monoxide, is a volume of acetic acid or an acetate-containing solution. The separation powered by the heat recovered from the electric arc furnace in this example involves one or more distillation steps, such as those conducted by distillation unit 402, for which the heat recovered from the electric arc furnace can be used.

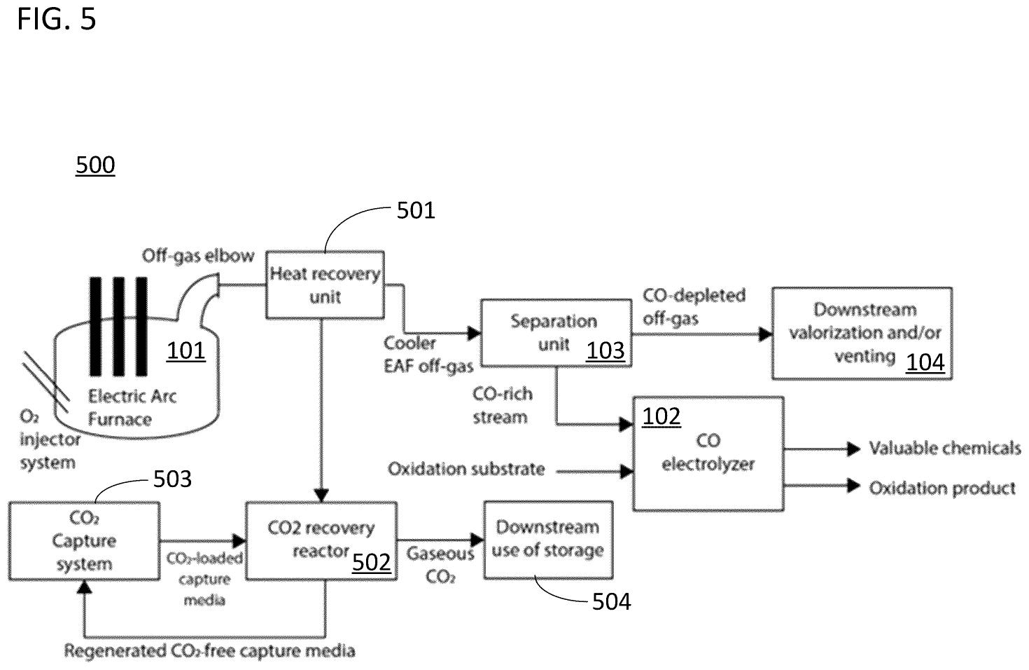

As stated previously, in specific embodiments of the inventions disclosed herein, heat recovered from an electric arc furnace can be used to power alternative systems for capturing carbon dioxide. For example, heat recovered from the electric arc furnace can be used for the regeneration of a media employed for the capture of additional carbon dioxide from another industrial exhaust source or from the air. Such media can include amine blend sorbents, adsorbents, or basic media such as potassium hydroxide. FIG. 5 illustrates a system 500 for valorizing a volume of carbon monoxide of an off-gas stream from an electric arc furnace 101 using a carbon monoxide electrolyzer 102 where heat from the off-gas as recovered by a heat recovery unit 501 is used to supply a carbon dioxide recovery reactor 502. The recovery reactor 502 uses heat to regenerate the media for carbon dioxide capture used by carbon dioxide capture system 503 and separate out isolated gaseous carbon dioxide for downstream use or storage 504. In specific embodiments, the downstream use or storage can involve converting the carbon dioxide to carbon monoxide, such as by using a reverse water gas shift reactor (RWGS reactor) and supplying the resulting carbon monoxide to carbon monoxide electrolyzer. In the case where a hydroxide-based solution is used for the capture of carbon dioxide, the recovery system used for the downstream regeneration of the hydroxide solution and release of the capture carbon dioxide in gaseous form implies the use of a calciner reactor in the place of carbon dioxide recovery reactor 502 where the heat recovered from the electric arc furnace is used to power the calciner reactor.

In specific embodiments of the inventions disclosed herein, a source of carbon dioxide in the system can be converted to carbon monoxide and this additional carbon monoxide can be supplied to the carbon monoxide electrolyzer. For example, the electric arc furnace carbon monoxide depleted off-gas may contain an appreciable amount of carbon dioxide (e.g., a proportion greater than 10%). The carbon monoxide depleted off-gas can then be purified and used as an input to a carbon dioxide to carbon monoxide conversion device. In alternative approaches such as those described herein in which a lime kiln is used in the production of calcium carbide, carbon dioxide from the lime kiln can be treated in the same manner.

Carbon dioxide to carbon monoxide conversion in accordance with embodiments of the inventions disclosed herein can be conducted with various reactors including a solid oxide electrolyzer, a low-temperature carbon dioxide electrolyzer, or a RWGS reactor, or a plasma-enhanced carbon dioxide to carbon monoxide catalytic reactor. In the case of a RWGS reactor, the RWGS reactor can be at least partially heated using the heat recovered from the electric arc furnace heat recovery unit. The RWGS reaction produces carbon monoxide and water from carbon dioxide and dihydrogen according to equation (3) presented below. This reaction is endothermic and thus thermodynamically enhanced at high temperature. The reaction is the opposite of the water gas shift reaction which is used in the production of dihydrogen. RWGS reactors are fed with a mix of carbon dioxide and dihydrogen at high temperature (100° C.-1000° C.) and mid pressure (1-30 bar). The H₂/CO₂ ratio of the feedstock for the reactor is usually on the order of between 0.9 and 2 according to the desired outlet syngas quality.

$$C {O}_{2} + {H}_{2} ↔ C O + {H}_{2} O Δ {H}_{r} = + 41.3 \frac{kJ}{mol} \qquad ( 3 )$$

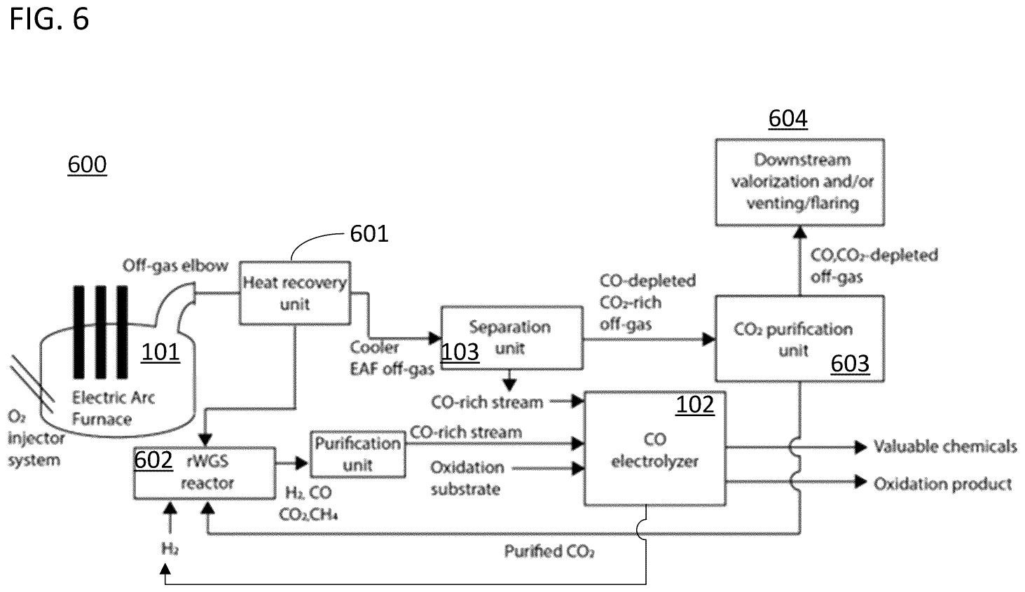

FIG. 6 illustrates a system 600 for valorizing a volume of carbon monoxide of an off-gas stream from an electric arc furnace 101 using a carbon monoxide electrolyzer 102 where heat from the off-gas as recovered by a heat recovery unit 601 is used to supply a RWGS reactor 602 for the conversion of carbon dioxide to carbon monoxide. In the illustrated example, the RWGS reactor 602 receives dihydrogen and purified carbon dioxide from carbon dioxide purification unit 603. The dihydrogen may be generated as a byproduct of the carbon monoxide electrolyzer for this reaction as shown. The carbon dioxide purification unit 603 operates on the carbon monoxide depleted off-gas from the electric arc furnace 101. The carbon monoxide and carbon dioxide depleted off-gas from the carbon dioxide purification unit 603 can then be sent for downstream valorization, venting, or flaring, using system 604. The RWGS reactor can use heat that is recovered from the electric arc furnace either directly or through a thermal storage system that harvests the heat from when the electric arc furnace is operating and applies the heat when the RWGS reactor is operating.

In specific embodiments of the inventions disclosed herein, the carbon monoxide depleted off-gas contains dihydrogen that is provided as at least part of the oxidation substrate to the carbon monoxide electrolyzer.

In specific embodiments of the inventions disclosed herein, the intermittency of the electric arc furnace due to the alternate operation steps including the loading of the furnace, start-up of the arc, melting, stopping of the arc, and removal of the slag and molten product is advantageously matched by an intermittent operation of the carbon monoxide electrolyzer. In specific implementations, this advantage is accentuated by the fact that the carbon monoxide electrolyzer can be operated at low temperature and has a start-up time from a full shutdown on the order of a minute or less. The carbon monoxide electrolyzer can be a low temperature carbon monoxide electrolyzer designed for this purpose. During operation of the electric arc furnace, carbon monoxide is released during the smelting and refining phases, which typically last between 30 and 60 minutes and account for 50-65% of the total duration of one operation cycle of the electric arc furnace. In specific implementations, the low-temperature carbon monoxide electrolyzer is intentionally operated with variable power consumption to match, fully or partially, the electronic arc furnace off-gas rate. The carbon monoxide electrolyzer current density can be varied in the minute range to match, fully or partially, the flow rate provided by the electric arc furnace process chain. Alternative routes to valorize carbon monoxide (such as but, not limited to, methanol synthesis and the Fischer-Tropsch process) have limited or no compatibility with power intermittency.

In specific embodiments of the invention, the resilience to intermittency of the carbon monoxide electrolyzer is advantageously used so that the carbon monoxide electrolyzer is operated asynchronously to the electric arc furnace to smooth the power consumption over time. An electric arc furnace typically draws 70-90% of its energy requirement during the melting phase which lasts 50-65% of the total duration of one phase of operation of the electric arc furnace. This concentrated energy requirement can cause significant stress on the electricity grid. Asynchronous operation of the carbon monoxide electrolyzer, which can minimize loading during the melting phase of the electric arc furnace and maximize loading during the charging, stopping and removal of the slag phases of the electric arc furnace operation, can considerably smooth the power consumption of the combined system. This is possible for low-temperature carbon monoxide electrolyzers with startup times on the range of less than a minute or minutes. In specific embodiments, the electrolyzer will have a startup time of less than 10 minutes.

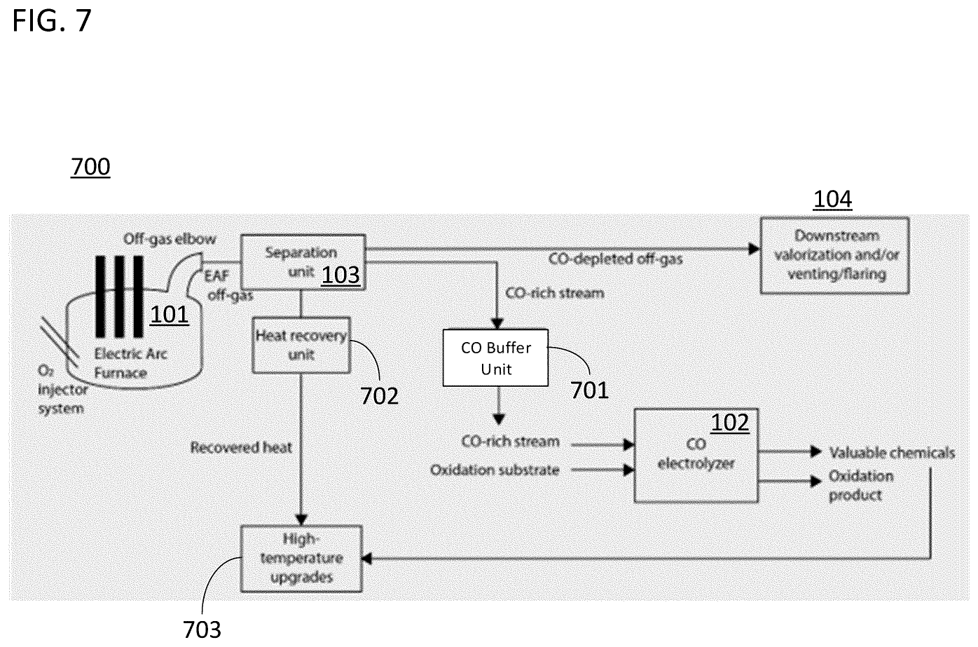

In specific embodiments of the inventions disclosed herein, a system can include a storage (e.g., an intermediate buffer tank) for off-gases. The storage can be used to accumulate the carbon monoxide and can include a compression system to limit the storage volume capability required. FIG. 7 illustrates a system 700 for valorizing a volume of carbon monoxide of an off-gas stream from an electric arc furnace 101 using a carbon monoxide electrolyzer 102 with a storage unit 701. As illustrated, storage unit 701 holds a volume of carbon monoxide from a carbon monoxide rich stream that has been produced by separation unit 103 operating on the off-gas of the electric arc furnace 101. The system also includes a heat recovery unit 702 which allows heat to be used by downstream processes on the chemicals generated by the carbon monoxide electrolyzer 102 in a high temperature upgrade unit 703. In the illustrated case, storage unit 701 does not include a compression system, but such a system could be included to increase the storage capacity of the storage unit. The storage unit could be a temporary buffer or a more long term storage tank. The flow of fluids between the blocks in the figure could be controlled by a system of valves. The valves and storage unit 701 could be designed to facilitate asynchronous operation of the electric arc furnace 101 and the carbon monoxide electrolyzer 102. The asynchronous operation may be particularly advantageous in that it more evenly distributes the power consumption of the overall system and because low-carbon emission power plants, such as nuclear power plants, operate more efficiently and more profitably when used at constant power.

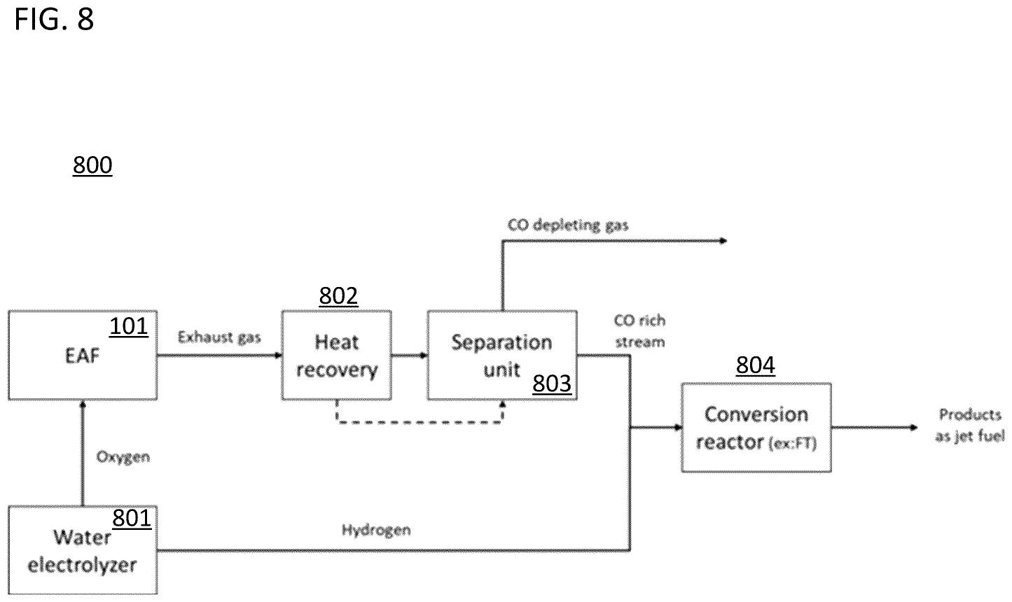

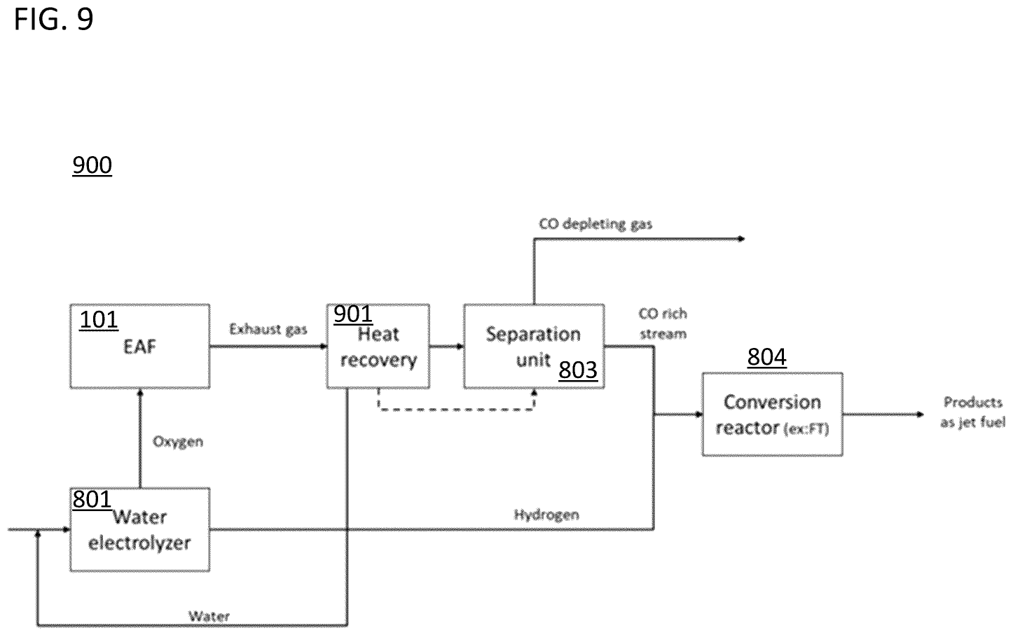

FIG. 8 illustrates a system 800 using an electric arc furnace 101 and a water electrolyzer 801. The system also includes a heat recovery unit 802 which provides heat to a separation unit 803. The separation unit 803 separates a carbon monoxide rich stream from the off-gas of the electronic arc furnace 101 and produces a stream of carbon monoxide depleted gas and the carbon monoxide rich stream. Downstream of the separation unit 803 the carbon monoxide rich stream is mixed with hydrogen produced by water electrolyzer 801. The carbon monoxide rich stream and dihydrogen can be combined in a conversion reactor 804 such as a Fisher Tropsch reactor to produce products such as syngas or jet fuel. The syngas quality (H₂/CO ratio) can be controlled by the hydrogen make-up. Typically, a ratio of two is used for a valorization into a Fischer Tropsch reactor producing liquid fuel such as kerosene or diesel. Methanol or methane, among other fuels, could also be produced from syngas. While not illustrated, in alternative approaches the co-produced oxygen coming from the water electrolyzer could be heated by the heat coming from the electronic arc furnace exhaust gas. The co-produced oxygen could also be introduced into the electronic arc furnace through dedicated oxygen injectors or be used by oxyfuel burners to chemically heat the furnace. In another embodiment, water condensate from the electronic arc furnace exhaust gas cooling step could be used as input in the water electrolyzer after purification. FIG. 9 illustrates a system 900 using an electric arc furnace 101 and a water electrolyzer 801. System 900 is similar to system 800 with the exception of the water from heat recovery unit 901 being supplied as an input to water electrolyzer 801.

Electric arc furnaces are increasingly used in the metal industry because they offer high quality products with reduced carbon emissions. Electric arc furnaces are used in multiple metal manufacturing processes, particularly in iron and steel production but also for the processing of titanium concentrates, molybdenum, or other various scrap metals.

In the case of steelmaking, one notable advantage of the electric arc furnace route is that it emits between 0.1 and 0.4 tons of carbon dioxide equivalent per ton of steel (in Europe) produced. In contrast, a much higher amount of 1.9 tons of carbon dioxide equivalent per ton of steel is typically produced using the conventional steelmaking route of using a blast furnace or a basic oxygen furnace. Using the electric arc furnace route thus offers significant environmental benefits and reduced carbon costs. The viability of the electric arc furnace process can be augmented by capturing and reducing the carbon emissions from the electric arc furnace off-gas.

An electric arc furnace used for steel production in accordance with this disclosure can comprise a refractory-lined bowl-shaped hearth where the scrap or direct reduced iron is placed and is then smelted to form a molten bath. The furnace can also comprise a refractory brick swivel roof that can be moved for the furnace to be loaded with the metal load. Alternatively, a charge door on the side of the furnace can be used to add metal and other compounds to the furnace. Multiple retractable carbon electrodes plunge through the roof into the furnace and create an arc when powered, heating the furnace to a temperature of 1500-3000° C. Additional heat may be provided through oxyfuel burners. Injectors can also penetrate the furnace to inject oxygen and or carbon sources into the furnace. The electric arc furnace off-gases can be evacuated from the furnace through exhaust systems that can be referred to as off-gas elbows or uptakes. Cooling panels and water-cooling systems can cool the roof and shell of the furnace. An eccentric bottom tap can be used to collect the melt. A foaming slag resulting from the reaction of scrap impurities with oxygen (e.g., oxygen injected through dedicated injectors), and oxidation of the solid carbon source employed (e.g., coke or coal) can be collected through a lateral slag door.

The smelting process carried out in the electric arc furnace proceeds intermittently through phases that notably include the loading of the furnace, start-up of the arc, melting, stopping of the arc, removal of the slag and molten product hampering the valorization of the electric arc furnace. The different phases of the process may last as little as a few minutes for a total cycle time in the range of half to a couple of hours. On average, phases last around 30 to 40 minutes. Although the composition of the electric arc furnace off-gas varies depending on the phase, it can comprise of at least 5% to over 60% of carbon monoxide during the smelting process. The temperature of the furnace can be over 1500° C. and can reach temperature over 2000° C. for small scale furnaces. The energy consumed to produce 1 ton of steel using the electric arc furnace is about 400-500 kWh.

The power input for an electric arc furnace used for steel production in accordance with this disclosure can range from 50 MVA to 200 MVA or higher. A mid-size steel electrical arc furnace can rely on a 60 MVA transformer to generate a high-current low-voltage source. The secondary voltage used to produce the electrical arc can range from 400 to 900 V and the secondary current during the melting phase can be greater than 40 kA. Three phase alternating current is generally used but direct current arc furnaces can also be found.

In specific embodiments of the inventions disclosed herein, a carbon monoxide electrolyzer is integrated downstream of an electric arc furnace producing CaC₂ and a dedusting and gas purification system. Electric arc furnace plants that produce CaC₂ generates a furnace gas rich in carbon monoxide but also in particulate matter that must be removed prior to further use. This requirement arises both from regulation to minimize environmental impact, and the conditions required to operate a downstream process to valorize the furnace off-gas. Unlike electric arc furnaces that primarily process scrap or other metallic burden, electric arc furnaces that produce CaC₂ take lime and carbon as process feedstock. In addition to CaO, the lime contains large amounts of other material such as, but not limited to, SiO₂, Fe₂O₃, Al₂O₃, MgO, P₂O₅, ZnO, and CuO. In some cases, the electric arc furnace takes waste plastic as a raw material, which may contain chlorine. As CaC₂ production proceeds, the lime and carbon react to form the desired CaC₂ material and a furnace off-gas of variable composition.

In specific embodiments of the inventions disclosed herein, a non-oxidative process is used to clean the gas stream of undesirable components other than carbon monoxide. In addition to CaC₂ and carbon monoxide, large amounts of dust particles are generated, along with undesirable and dangerous gases such as phosphine, carbonyl sulfide, chlorine gas, and hydrogen cyanide. Other gases produced include but are not limited to hydrogen, nitrogen, methane, and carbon dioxide. If an off-gas having the above characteristics were fed directly to a carbon monoxide electrolyzer, the dust and undesirable gases produced by the electric arc furnace would negatively impact the efficiency and lifetime of the carbon monoxide electrolyzer, in some cases irreversibly. Chlorine gas, for example, will corrode the steel or titanium material used in the carbon monoxide electrolyzer, leading to higher expenditures for maintenance and replacement.

Most methods used by electric arc furnace process operators to clean-up the furnace off-gas are oxidative in nature and unsuitable for integration with carbon monoxide electrolysis. After de-dusting, operators of electric arc furnaces typically clean up the furnace-off-gas after dedusting by flaring the mixture with air to produce a mixture of gases such as N₂, SOx, NOx, P₂O₅, H₂O, HCl, and carbon dioxide. However, an oxidative process such as flaring releases the energy embedded in carbon monoxide to produce carbon dioxide, squandering an opportunity to use the carbon monoxide in an electrolyzer to produce value-added chemicals. If they are not removed, the acid equivalents present in the gas stream also reduce the pH of the carbon monoxide electrolyzer, increasing the cost of operation. The inclusion of a non-oxidative separation process has not been obvious because most routes to upgrade carbon monoxide to value-added chemicals (such as methanol synthesis, the Sabatier reaction to produce methane, or the Fischer Tropsch process to produce hydrocarbons) are relatively insensitive to the presence of carbon dioxide and/or other acid gases.

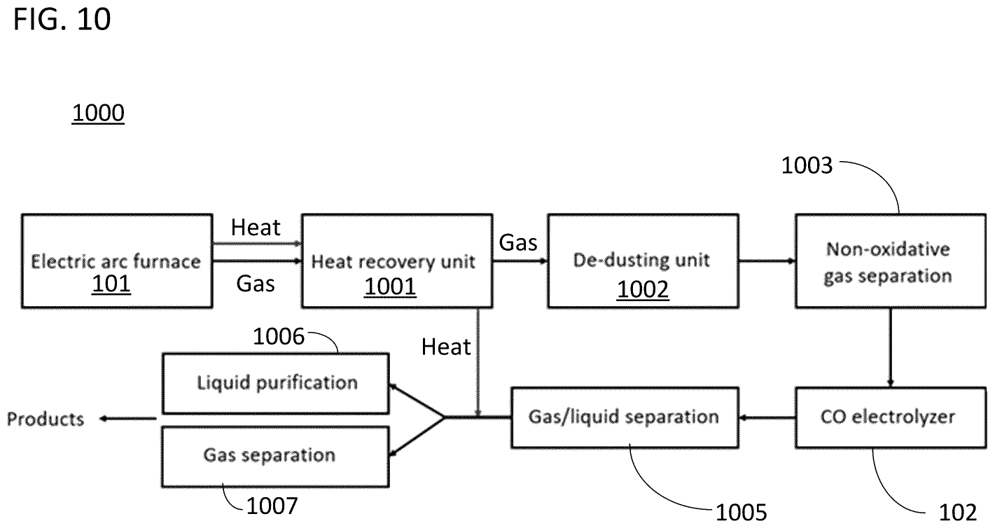

A non-oxidative separation technology, such as, but not limited, to absorption, adsorption, or membrane separation, can be employed to recover the carbon monoxide from an electric arc furnace. In non-oxidative separation technologies, care is taken to minimize the oxidation of reduced compounds in the gas mixture, such as carbon monoxide and dihydrogen. FIG. 10 illustrates a system 1000 for valorizing a volume of carbon monoxide of an off-gas stream from an electric arc furnace 101 using a carbon monoxide electrolyzer 102 with a non-oxidative gas separation unit 1003 used to treat the off-gas before it is provided to the carbon monoxide electrolyzer 102. As illustrated, the system includes a de-dusting unit 1002 downstream of a heat recovery unit 1001 and upstream of the non-oxidative gas separation unit 1003. For example, after leaving the de-dusting unit 1002, the treated furnace off-gas can be treated with a pressure swing adsorption system and removal of acid-gas via absorption. The purified carbon monoxide rich stream can then be supplied to the carbon monoxide electrolyzer. The resulting stream can then be sent to a gas/liquid separation unit 1005 to split the stream into liquid and gaseous products. The liquid products can then be treated by a liquid purification unit 1006 and the gaseous products can then be treated by a gas separation unit 1007.

In specific embodiments of the invention, the heat contained within the EAF off-gas cooled prior to having the volume of carbon monoxide delivered to the carbon monoxide electrolyzer. The off-gas can be cooled using a non-oxidative cooling process. In specific embodiments of the invention, the heat is advantageously recovered using a non-oxidative heat exchange and/or heat storage system, while minimizing the conversion of the carbon monoxide to carbon dioxide and provisioned into processes upstream or downstream of the carbon monoxide electrolyzer. One barrier to valorizing the off-gas of an EAF is the high temperatures associated with the off-gas. The EAF off-gas can be cooled by mixing the EAF exhaust with air. This cools the off-gas and oxidizes the carbon monoxide within the off-gas into carbon dioxide. However, conversion of the carbon monoxide to carbon dioxide is undesirable in specific embodiments disclosed herein because using carbon dioxide as a feedstock for a carbon monoxide electrolyzer reduces its process efficiency. Alternatively, to preserve carbon monoxide content in the EAF off-gas for use in the carbon monoxide electrolyzer, the heat embodied within the EAF off-gas can be harvested or removed using a process that does not induce oxidation of carbon monoxide into carbon dioxide, such as, but not limited to, a series of heat exchangers, thermal masses, and/or thermal batteries. The cooling can be conducted by contacting the off-gas with a thermal storage system such as a thermal battery or brick stack. The harvested heat can be advantageously used in an element of the overall process chain, such as gas separation or liquid production distillation, to reduce heat input into the overall process, or be valorized in a different manner, such as, but not limited to, being use as municipal or industrial heat or to generate electricity. For example, the heat can be used to capture carbon dioxide such that the thermal storage system is used to capture carbon dioxide. To be suitable for valorization within the process chain, the EAF off-gas can be sufficiently cooled as to be compatible with the next, immediate component of the process chain, which may include but is not limited to a gas separation unit to purify the carbon monoxide, a dedusting unit, or the carbon monoxide electrolyzer itself. In the case of integration to the separation unit, the EAF off-gas is cooled to a temperature range of 25 to 350° C., depending on the technology used. For the carbon monoxide electrolyzer to function effectively, feedstock gases are provisioned in the range of 0 to 100° C. Prior approaches have not utilized a cooling process for an EAF off-gas because existing methods to valorize the EAF off-gas are relatively insensitive to high temperatures (such as but not limited to flaring) or to the presence of carbon dioxide (such as but not limited to Fischer-Tropsch and methanol synthesis).

In a specific embodiment of the invention, a carbon monoxide electrolyzer is designed to mitigate cost and improve the productivity of a CaC₂ electric arc furnace process. In some cases, the rate of CaC₂ production in a facility is limited by the ability to sufficiently flare the furnace off-gas and/or remove the dust particles from the furnace off-gas. Because of regulations, these cleanup processes must be employed but ultimately represent a cost to an electric arc furnace operator from which limited additional revenue can be derived. Expanding the capacity of an electric arc furnace plant to treat and clean up more furnace-off-gas requires substantial capital expenditures, in part because of the equipment and expertise required to handle a highly combustible and toxic gas mixture. In contrast, integrating a carbon monoxide electrolyzer downstream of the electric arc furnace off-gas cleanup processes provides an opportunity to recoup the costs associated with the off-gas cleanup. Compared to using the furnace off-gas to generate heat, using the carbon monoxide generated in the electric arc furnace to supply a carbon monoxide electrolyzer can produce more value and lead to higher net margins.

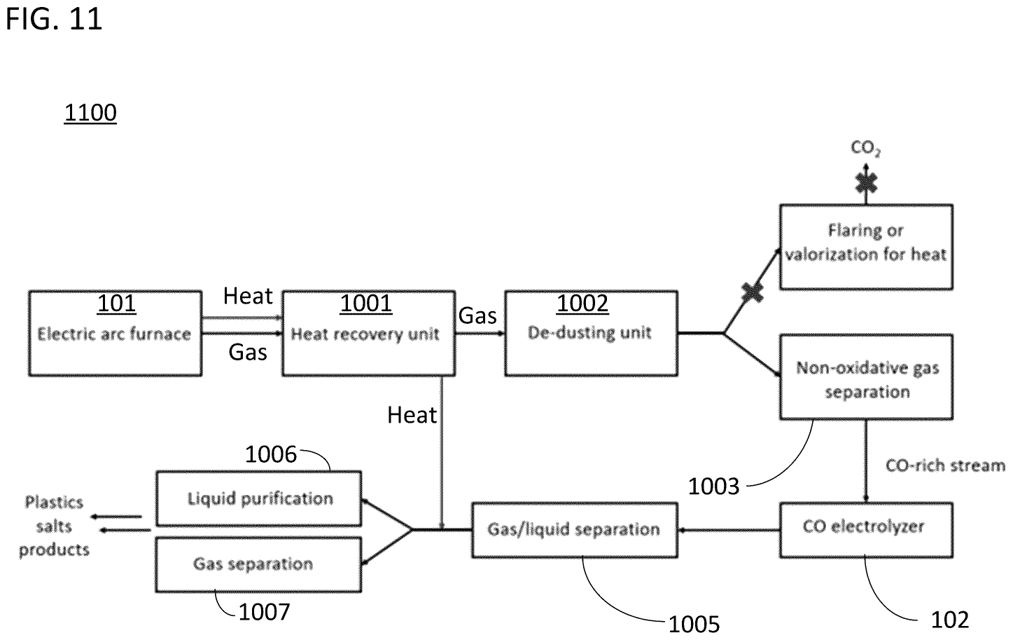

Integrating a carbon monoxide electrolyzer downstream of a CaC₂ electric arc furnace process also provides a novel cost-effective mechanism for the operators of an electric arc furnace to reduce their carbon emissions and mitigate regulatory risk. The chemistry of the CaC₂ process imposes a fundamental 1:1 stoichiometry between the amount of CaC₂ and carbon monoxide produced. Despite its many critical uses, at present there are no viable alternatives to produce CaC₂ without using coke or coal as a feedstock. Because the stoichiometrically produced carbon monoxide is typically flared or valorized for heat and produces carbon dioxide, the operator of an electric arc furnace process must then capture and sequester these direct carbon dioxide emissions for the process to be compliant with net-zero carbon emissions standards or regulations. Additional indirect emissions arise from the necessity of providing heat and electricity to the process, typically from burning fossil fuels. Operators of such a system have limited opportunities to derive value from the sequestered carbon dioxide, representing a cost and exposure to regulatory risk. In contrast, converting the carbon monoxide in the furnace off-gas using a carbon monoxide electrolyzer (and critically, before its combustion to carbon dioxide) effectively utilizes the energy embedded in the carbon monoxide to produce chemicals with more value (once sold) than valorizing carbon monoxide for heat. This combined process provides a route to recoup the cost of carbon capture and sequestration. Moreover, additional processes downstream of the carbon monoxide electrolyzer that convert the carbon monoxide electrolyzer products into long-lived solids (such as but not limited to plastics and salts) thus provides a viable mechanism for the electric arc furnace industry to provide feedstocks for low or carbon-neutral chemicals and materials. FIG. 11 illustrates a system 1100 for valorizing a volume of carbon monoxide of an off-gas stream from an electric arc furnace 101 using a carbon monoxide electrolyzer 102 with a non-oxidative gas separation unit 1003 used to treat the off-gas before it is provided to the carbon monoxide electrolyzer 102. FIG. 11 also illustrates how the flaring or valorization of carbon monoxide for heat and the concomitant emission of carbon dioxide can be eliminated.

Opportunities for sustainable low-carbon carbon monoxide electrolysis have been hindered by a lack of technology maturity and a lack of a sustainable source of carbon monoxide and process heat. At present, the dominant and lowest-cost source of carbon monoxide for industrial processes is the steam methane reforming (SMR) process to produce carbon monoxide and dihydrogen, which also produces large amounts of carbon dioxide by reacting methane with water. One reason it has not been obvious to integrate an electric arc furnace system with carbon monoxide electrolyzers is the fact that electric arc furnace off-gases have largely been valorized for heat or flared as waste, with oxidative processes being applied for gas cleanup. Another reason this integration has not been obvious is that electric arc furnace processes and operators are generally located in geographies with easy access to coal and/or coke but limited access to natural gas, which is the primary feedstock required to produce carbon monoxide from SMR. Today, large-scale carbon-monoxide-consuming industrial processes are also generally located in geographies with ready access to natural gas-derived carbon monoxide. The requirement of additional process heat (which an electric arc furnace can provide) used to purify carbon monoxide feedstock streams going into a carbon monoxide electrolyzer and the product streams leaving the carbon monoxide electrolyzer imposes an additional cost to operating a carbon monoxide electrolyzer. Because the economic viability, performance, and emissions intensity of carbon monoxide electrolyzers is very sensitive to the composition of the electrolyzer gas supply and the source of process heat, supplying a carbon monoxide electrolyzer with waste carbon monoxide from a CaC₂ electric arc furnace process can substantially lower the feedstock cost of the carbon monoxide electrolyzer, provide a route to cost-effectively reduce the emissions footprint of the electric arc furnace process, and enable a new low-emissions chemicals production technology that would otherwise be difficult to accomplish with each technology separately.

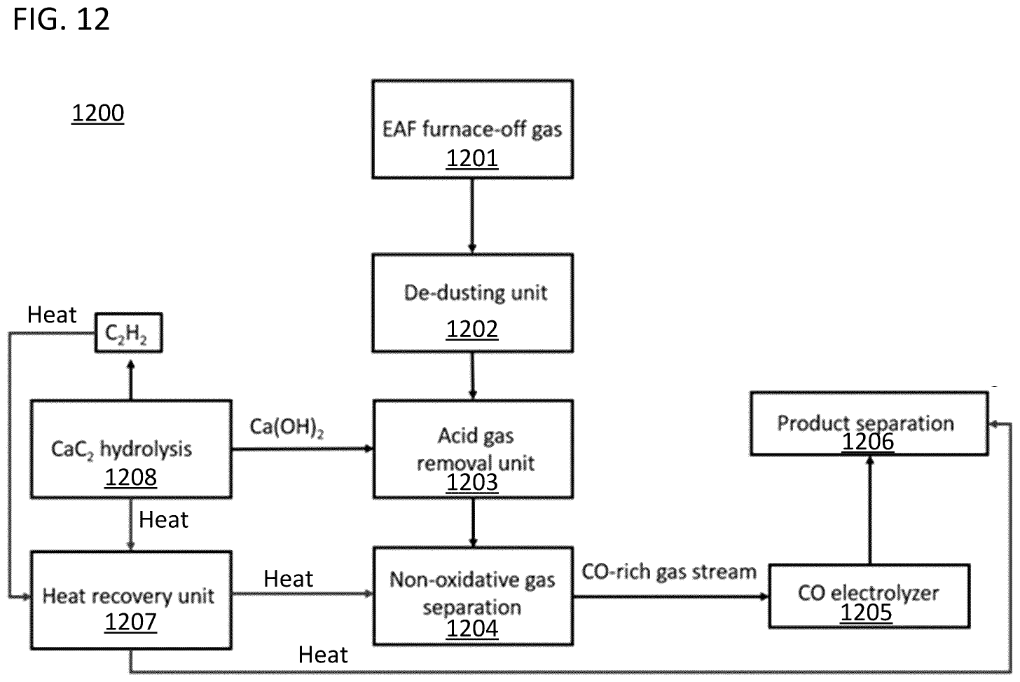

In certain embodiments of the invention, a carbon monoxide electrolyzer is integrated with a broader CaC₂ to acetylene process chain to recoup process heat and alkaline equivalents required for sustainable operation. A block diagram 1200 of an according system integration is illustrated in FIG. 12. Acetylene is produced from CaC₂ via hydrolysis, according to eq (4):

$$C a {C}_{2} + {H}_{2} O ↔ {C}_{2} {H}_{2} + C {a ( O H )}_{2} Δ {H}_{r} = - 128.4 \frac{kJ}{mol} \qquad ( 4 )$$

The hydrolysis of CaC₂ to acetylene and Ca(OH)₂ produces a large amount of heat that can be recovered using heat exchangers as the acetylene departs the hydrolysis reactors. This is illustrated by the heat lines departing the CaC₂ hydrolysis block 1208 and the acetylene block leading to heat recovery unit 1207. The recovered heat can be synergistically supplied to processes required to operate the carbon monoxide electrolyzer, including upstream purification of the electrolyzer feed gas, such as non-oxidative gas separation unit 1204, and downstream purification of the gas and/or liquid products departing the carbon monoxide electrolyzer, such as product separation unit 1206. Recovering the excess heat from the CaC₂ hydrolysis process has not previously been conducted because it is not economical and because of the high temperatures required in the upstream process chain (such as lime drying and the reaction of coke with lime).

Upon hydrolysis, each equivalent of CaC₂ produces an equivalent of Ca(OH)₂, which is typically treated by process operators as a waste and stockpiled near CaC₂ and acetylene production facilities. The generated Ca(OH)₂ is typically not valorized as a product because impurities arising from the solid CaC₂ are entrained in the solid product. However, the Ca(OH)₂ produced by the hydrolysis can be advantageously used as a sacrificial sorbent material to scrub the process gas stream before it enters the carbon monoxide electrolyzer. This is illustrated in FIG. 12 by Ca(OH)₂ being supplied to acid gas removal unit 1203. The acid gas removal unit can remove carbon dioxide from the electronic furnace off-gas 1201 after a de-dusting unit 1202 has removed the dust from the off-gas. Ca(OH)₂ reacts exothermically with acid gases such as CO₂ and H₂S via reactions 5 and 6.

$$C {a ( O H )}_{2} + C {O}_{2} ↔ C a C {O}_{3} + {H}_{2} O - 69.1 \frac{kJ}{mol} \qquad ( 5 )$$

Ca(OH)₂+H₂S↔CaS+2H₂O (6)

When the Ca(OH)₂ generated by the CaC₂ hydrolysis process is fed into an acid gas removal unit, it can reduce the energy and material demand of the process. As illustrated, process heat generated by the hydrolysis of CaC₂ is harvested using a heat recovery unit 1207 to supply the gas separation processes along the process chain. To prevent contamination and deactivation of the acid-gas and non-oxidative gas separation units (e.g. with membranes or with adsorption) with metals and/or impurities present in the Ca(OH)₂ added, the Ca(OH)₂ can be packed into a separate solid absorbent bed prior to entering the gas purification process chain.

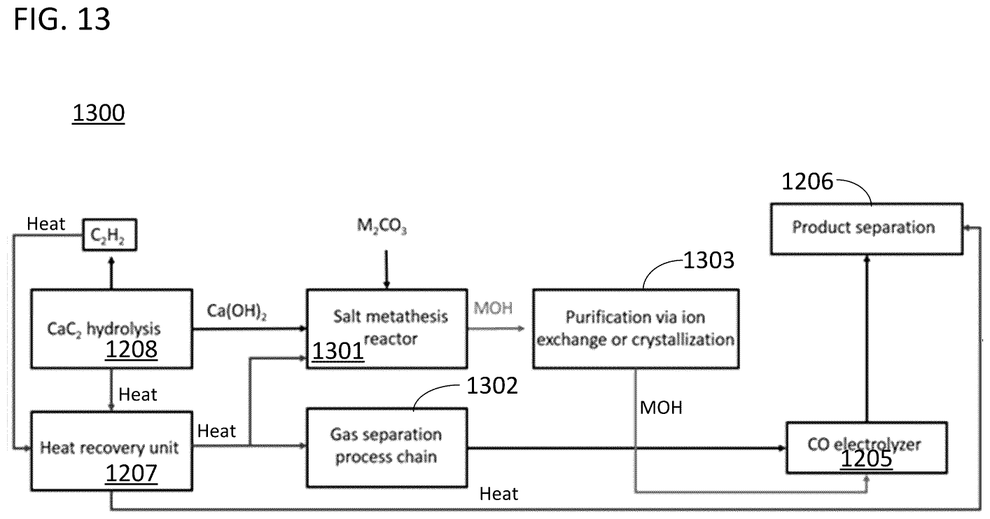

In cases where the Ca(OH)₂ generated is sufficiently pure, such as from a process with high lime purity, alkaline equivalents used to supply the carbon monoxide electrolyzer can be sourced from the Ca(OH)₂ wastes via salt metathesis with Na₂CO₃, K₂CO₃, Li₂CO₃, or Cs₂CO₃. FIG. 13 illustrates a system 1300 in which the calcium hydroxide wastes from calcium carbonate hydrolysis in the calcium carbonate hydrolysis block 1208 are used to produce an electrolyte for operation of a carbon monoxide electrolyzer 1205. On a molar basis, such alkali metal carbonates are generally of lower value than their hydroxide counterparts, and thus represent an economical way to generate metal hydroxide equivalents necessary for operating the carbon monoxide electrolyzer. In these embodiments, the Ca(OH)₂ can be boiled with alkali metal carbonate in a salt metathesis reactor (such as salt metathesis reactor 1301) to induce ion exchange, followed by purification of the MOH product using ion exchange, adsorption, crystallization or other methods (such as in purification unit 1303) to improve the purity of the metal hydroxide product.

Ca(OH)₂+M₂CO₃↔CaCO₃+2MOH (7)

Ion exchange can be performed using an ion exchange resin containing charged functional groups that are capable of adsorbing cationic species, such as, but not limited to, Nafion, Dowex, or Chelex resin. The MOH product can also be purified via adsorption, e.g., by flushing an aqueous solution of MOH through an activated sorbent bed composed of materials such as, but not limited to, activated carbon, molecular sieves, zeolites, or some other high adsorption capacity material. The purified MOH product (either dissolved in water or as a solid) can then be added to the carbon monoxide electrolyzer process chain (e.g., by being used as an electrolyte in carbon monoxide electrolyzer 1205 as it operates on the carbon monoxide rich stream that is the process of gas separation process chain 1302). In addition to the energy and material savings for the carbon monoxide electrolysis process chain, these process integrations are advantageous for the broader CaC₂-to-acetylene process because they provide a route to valorize the Ca(OH)₂ waste generated by the CaC₂ hydrolysis, and also reduces the barrier for valorizing the furnace off-gas using carbon monoxide electrolysis.

The cycle of an electric arc furnace can consist of various phases. The cycle can include a charging phase in which the raw materials such as scrap metal, pig iron, and alloys are charged into the furnace (on the example of an electric arc furnace in the metal fabrication industry). The duration of the charging phase typically ranges from 10 to 30 minutes. The cycle can include a melting phase in which the electric arc is created between the electrodes and the materials are heated to their melting point, causing them to melt. The duration of the melting phase typically ranges from 30 to 60 minutes. This phase draws 70-90% of the energy necessary to produce the steel. The cycle can include a refining phase. Once the materials are molten, the refining phase begins where the impurities are removed using various methods such as oxygen injection or fluxing. The duration of the refining phase typically ranges from 10 to 30 minutes. The cycle can include a tapping phase. Once the desired composition and temperature are achieved, the molten metal is tapped from the furnace and transported to the next process, such as casting or further refining. The duration of the tapping phase typically ranges from 10 to 20 minutes. The cycle can include a slagging phase. The remaining slag can be removed from the furnace and cooled for disposal or reuse. The duration of the slagging phase typically ranges from 10 to 20 minutes. These phases may vary depending on the specific requirements and operation of the electric arc furnace.

The intermittency of an electric arc furnace can cause significant stress on an electrical grid especially for large furnaces with power >50 MVA. The intermittency can however be compensated by advantageously operating a carbon monoxide electrolyzer asynchronously. In the case of steel production, the electric arc furnace energy consumption to produce one ton of steel is 400-500 kWh. This process generates 30-40 kg of CO per ton of steel. Assuming the energy requirements of the carbon monoxide electrolyzer is 10-15 kWh per kg of carbon monoxide converted, the energy consumption of the carbon monoxide electrolyzer is 300-600 kWh per ton of steel, which fairly matches the energy consumption of the electric arc furnace.

In the context of calcium carbide production, the electric arc furnace energy consumption to produce one ton of CaC₂ is 4000 kWh. This process generates 437 kg of CO per ton of CaC₂ produced. Thus, the energy consumption of a carbon monoxide electrolyzer coupled to the calcium carbide plant is 4300-6500 kWh per ton of CaC₂. This value matches or slightly exceeds the energy consumption of the electric arc furnace.

Considering that the melting phase is 70-90% of the electric arc furnace energy requirements and 50-65% of the duration of one operation cycle, a combined system consisting of an electric arc furnace and a carbon monoxide electrolyzer can be operated at almost constant power by reducing the load on the carbon monoxide electrolyzer during the melting phase and increasing the load during the other phases. Such an operation enables significant and even total conversion of the carbon monoxide molecules contained in the off-gas stream, both for the steel case and the calcium carbide case. This is possible for low temperature carbon monoxide electrolyzers whose capacity can be varied from 0 to 100% on the order of a few minutes.

The load on the carbon monoxide electrolyzer can be controlled in an automatized way by different means such as, but not limited to, regulating the load on the carbon monoxide electrolyzer depending on the load of the electric arc furnace, regulating the load on the carbon monoxide electrolyzer based on measuring one or several of the operation parameters of the electric arc furnace such as, but not limited to, pressure, carbon monoxide partial pressure, mass flow or volume flow of the off-gas stream.

A carbon monoxide electrolyzer in accordance with embodiments disclosed herein can have various architectures for the conversion of carbon monoxide into valuable chemicals. The electrolyzer can include an anode area and a cathode area. The carbon monoxide can be provided to the anode area. The useful chemicals can be produced in the cathode area, in the anode area, or in a separating area located between the cathode area and the anode area of the electrolyzer. The electrolyzer can be a single planar electrolyzer. The electrolyzer can be a stack of cells. The cells in the stack can utilize bipolar plates. The bipolar plates can be charged to initiate reactions within the reactor. The electrolyzer can also be a filter press electrolyzer or a tubular electrolyzer.

In specific embodiments of the inventions disclosed herein, the electric arc furnace is advantageously integrated with a carbon monoxide electrolyzer comprising a cathode area where carbon monoxide reduction takes place according to equation 8 below and an anode area where an oxidation reaction takes place on an oxidation substrate. The oxidation substrate can be water, dihydrogen, halides, organic waste or any other oxidation substrate. For example, the oxidation can involve water oxidation or dihydrogen oxidation according to equations 9 and 10 below respectively.

XCO+(x+y−z)H₂O+(2x+y−2z)e⁻ [symbol] CₓH_{y}O_{z}+(2x+y−2z)OH⁻ (8)

2H₂O [symbol] 4H⁺+4e⁻+O₂ (9)

H₂ [symbol] 2H⁺+2e⁻ (10)

Both the carbon monoxide and the oxidation substrate can be mixed with additive chemicals to alter the characteristics of the reactor and change the characteristics of the chemicals produced by the electrolyzer. For example, water and carbon monoxide can be combined to form a cathodic input fluid for the electrolyzer, while an oxidation substrate such as water or dihydrogen is provided on another connection coupled to an anode input of the electrolyzer.

The chemicals produced by the electrolyzer can vary in different embodiments of the invention. The chemicals can be separated using a separating element such as a trap for liquid chemicals on the anodic or cathodic output of the electrolyzer or a separating area between the cathode area and anode area which has its own output from the electrolyzer. The chemicals produced can be removed from the electrolyzer in solid or gaseous form and can be removed from the cathodic or anodic output streams on the cathode or anode outputs of the electrolyzer, or from a separate output from a separating layer. Examples of such a separating layer are provided below. A single electrolyzer can produce chemicals in both gaseous and liquid forms simultaneously. Accordingly, the volume of chemicals generated could include at least one of a volume of hydrocarbons, a volume of organic acids, a volume of alcohols, a volume of olefins, and a volume of N-rich organic compounds, where the chemicals are in gaseous or liquid form. For example, the volume of generated chemicals could include a volume of gaseous hydrocarbon and a volume of liquid alcohol. As another example, the volume of generated chemicals could include a volume of gaseous hydrocarbons and a volume of organic acids. In a specific embodiment, the main targeted products are ethylene (in the gaseous product stream) and acetic acid/acetate (in the liquid product stream). In another embodiment, the main targeted product is propanol (in the liquid product stream).

In specific embodiments of the invention, the anodic reaction of the carbon monoxide electrolyzer is that of water and the oxygen produced is reinjected through the oxygen injectors in the electric arc furnace to help with the formation of the foamy slag and the decarburization of the molten bath and oxidation of impurities.

In other embodiment, the anodic reaction used at the anode is that of dihydrogen and such dihydrogen can be supplied from a low-carbon-footprint hydrogen-generating system.

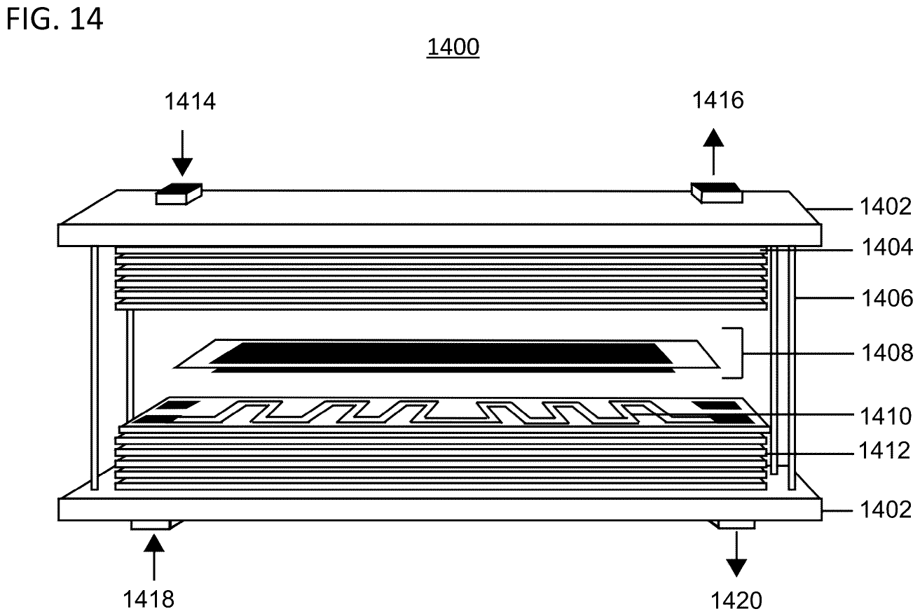

The carbon monoxide electrolyzer used in accordance with this disclosure can comprise one or more electrocatalytic cells positioned on top or next to one another to increase the surface available for the reaction. They can be stacked on top of one another, and such stacks can also be parallelized. These cells may be connected in series or in parallel. Many different cell and stack configurations can be used for the electrolyzers in accordance with this disclosure. FIG. 14 provides a diagram of an electrolyzer 1400 for explanatory purposes. The methods and systems disclosed herein are broadly applicable to electrolyzers that can receive carbon inputs such as carbon monoxide generally and electrolyzer 1400 is provided as a nonlimiting example of one such an electrolyzer.

FIG. 14 illustrates an electrolyzer 1400 which can be used for the electrolysis of carbon monoxide in the form of a stack in accordance with specific embodiments of the invention disclosed herein. The electrolyzer 1400 includes end plates such as 1402, monopolar plates such as 1404, rigid bars such as 1406, a membrane electrode assembly (MEA) such as 1408 or any form of catalytic core, a flow field such as 1410, and bipolar plates such as 1412. Again, while the example of an MEA is being provided, this is only an example, and electrolyzers with any form of catalytic cores can be used in accordance with the embodiments disclosed herein. Additionally, the stack of electrolyzer 1400 includes an inlet 1414 and an outlet 1416 for an anodic stream, as well as an inlet 1418 for a cathodic stream and an outlet 1420 for the cathodic stream. The polar plates, such as monopolar plate 1404 and bipolar plate 1412 can be part of the cells in the stack. The stack can also comprise gasketing, sealing of any shape, insulating layers and materials that have not been represented in FIG. 14 for clarity.

In an electrolysis stack, subsequent cells can be physically separated by bipolar plates (BPPs), such as bipolar plate 1412 in FIG. 14, that can ensure mechanical support for each of the electrolysis cells on each side of the BPP. BPP can also ensure electrical series connection between subsequent electrolysis cells and introduce/remove the reactants/products respectively. At the end of the stack, only one side of the plate can be in contact with the terminal cell; it is then called a monopolar plate, such as monopolar plate 1404 in FIG. 14. At the extremities of the stack, current collectors can allow connection to an external power supply, which can also be used, among other elements, for electrical monitoring of the stack. The stack can be assembled within a stack casing allowing its mechanical support and compression, as well as provisioning and transporting the reactant and product streams to and from the stack. The stack casing can comprise end plates that ensure electrical isolation of the stack and provide the inlet and outlets for the reactant and product streams. Alternatively, insulator plates can be placed between end plates such as 1402 and the monopolar plate such as 1404 to ensure electrical insulation of the stack versus the stack casing depending on the material of the end plate.

The carbon monoxide electrolyzers can take as an input, a cathodic input stream (e.g., stream enriched in carbon monoxide) and an anode input stream. The cathodic input stream can be provided to an inlet such as inlet 1418. The anodic input stream can be provided to an inlet such as inlet 1414. The cathodic stream and anodic stream can flow through the stack from the inlets to the outlets and be distributed through the flow channels, such as flow channel 1410 of each cell to each cathodic and anodic area separately. The anodic stream and cathodic stream would flow through separate channels on either side of the cell. Alternatively, at least one of the cathodic and anodic streams may be provided to each cell individually instead of through a connection crossing all the plates. In this case, each cell has a dedicated fluid inlet and outlet for this cathodic and/or anodic stream. The nature of the anodic stream can be determined by the nature of the targeted oxidation reaction (such as, but not limited to, water oxidation, dihydrogen oxidation, chloride oxidation, halide oxidation, hydrocarbon oxidation, waste organic oxidation). When electrically powered, the carbon monoxide electrolyzer carries out the concomitant reduction of carbon monoxide and oxidation of the chosen oxidation substrate to produce added-value chemicals such as hydrocarbons, organic acids and/or alcohols and/or N-containing organic products in the output cathodic stream separated from the anodic stream where the oxidation products are specifically collected. For example, the generating of chemicals using carbon monoxide and the electrolyzer could involve supplying the volume of carbon monoxide to a cathode area of the electrolyzer as a cathodic input fluid and supplying a volume of water to an anode area of the electrolyzer as an anodic input fluid.

In specific embodiments of the invention, the anode area could comprise an anodic catalyst layer able to oxidize a substance to produce a product and protons. The catalyst can comprise one or more of: molecular species, single-metal-site heterogeneous compounds, metal compounds, carbon-based compounds, polymer electrolytes (also referred to as ionomers), metal-organic frameworks, metal-doped covalent organic framework, or any other additives. The molecular species can be selected from metal porphyrins, metal phthalocyanines or metal bipyridine complexes. The metal compound can be under the form of metal nanoparticles, nanowires, nano powder, nanoarrays, nanoflakes, nanocubes, dendrites, films, layers, or mesoporous structures. The single-metal-site compounds can comprise a metal-doped carbon-based material or a metal-N—C-based compound. Anodic catalyst species used for this purpose could include, but are not limited to, metals and/or ions of: Ir, Co, Cu, Ni, Fe, Pt, Rh, Re, Ru, Pd, Os, Mo, and mixture and/or alloys thereof. For example, the anodic catalyst could be Ni such that the electrolyzer assembly included a nickel-based anode. The polymer electrolyte can be selected out of the same materials as the one used for the described membranes. The carbon-based compounds can comprise carbon nanofibers, carbon nanotubes, carbon black, graphite, boron-doped diamond powder, diamond nanopowder, boron nitride, or a combination thereof. The additives can be halide-based compounds including F, Br, I, and Cl. The additives can be specifically dedicated to modify hydrophobicity such as treatment with polytetrafluoroethylene (PTFE), Nafion or another hydrophobic polymeric ionomer additive, or carbon black. The anodic catalyst may be chosen to tune the performance and net product stream of the electrolyzer by choosing catalysts that are more or less capable of anodic alcohol oxidation to the corresponding carboxylic acid, aldehyde, or carbon dioxide.

The anodic catalyst may be deposited onto a gas diffusion layer or a porous transport layer or any other support that facilitates the diffusion of gas from the interface of the anode to a purified gas stream separated from the cathodic stream. The anode area could also include a gas diffusion layer with one or more separators such as, but not limited to, membranes, polymeric materials, diaphragm, and inorganic materials on its borders as described below.

In specific embodiments of the invention, the cathode area could comprise a catalyst layer able to reduce a substance (e.g., carbon monoxide) to generate value-added hydrocarbons/alcohols/organic acids. The catalyst can comprise one or more: molecular species, single-metal-site heterogeneous compounds, metal compounds, carbon-based compounds, polymer electrolytes (also referred to as ionomers), metal-organic frameworks, or metal-doped covalent organic frameworks or any other additives. The molecular species can be selected from metal porphyrins, metal phthalocyanines or metal bipyridine complexes. The metal compound can be under the form of metal nanoparticles, nanowires, nano powder, nanoarrays, nanoflakes, nanocubes, dendrites, films, layers or mesoporous structures, with precisely chosen particle sizes to control performance. The single-metal-site compounds can comprise a metal-doped carbon-based material or a metal-N—C-based compound The cathode catalyst may be made of a metal or metal ion from metals such as, but not limited to, Cu, Ag, Au, Zn, Sn, Bi, Ni, Fe, Co, Pd, Ir, Pt, Mn, Re, Ru, La, Tb, Ce, Dy, or other lanthanides and mixture and/or alloys thereof. For example, the cathodic catalyst could comprise Cu such that the electrolyzer assembly included a copper-based cathode. The polymer electrolyte can be selected out of the same materials as the one used for the described membranes. The carbon-based compounds can comprise carbon nanofibers, carbon nanotubes, carbon black, graphite, boron-doped diamond powder, diamond nanopowder, boron nitride, or a combination thereof. The additives can be halide-based compounds including F, Br, I, or Cl. The additives can be specifically dedicated to modify hydrophobicity such as treatment with PTFE, Nafion or another hydrophobic polymeric ionomer additive, or carbon black. The cathode may further comprise a catalyst layer on a gas diffusion layer, a porous transport layer, or any other support, which encourages the diffusion of the gas from a stream to the surface of the catalyst, as well as allowing the release of non-reacted/product gases. The cathode area could also include a gas diffusion layer with one or more separators such as, but not limited to, membranes, polymeric materials, diaphragms, and inorganic materials on its borders as described below. The loading of catalyst and additives on the gas diffusion layer can be precisely chosen to favor certain performance characteristics, such as differences in voltage, conductivity, carbon monoxide mass transport rate, product selectivity, and stability.

In specific embodiments of the invention, the porous support for either the anode area, the cathode area, or both, can be selected from carbon-based porous supports or metal-based porous material, or a combination. The carbon-based porous support can be based on carbon fibers, carbon cloth, carbon felt, carbon fabric, carbon paper, molded graphite laminates and the like or a mixture thereof. The carbon-based porous support can be a gas diffusion layer with or without microporous layer. Such carbon-based support can be in particular chosen in the among the following list: Sigracet 39AA, Sigracet 39BC, Sigracet 39BB, Sigracet 39BA, Sigracet 36AA, Sigracet 36BB, Sigracet 35BC, Sigracet 35BA, Sigracet 29BA, Sigracet 28BB, Sigracet 28AA, Sigracet 28BC, Sigracet 25BC, Sigracet 22BB, Sigracet 35BI, Toray papers, Toray THP-H-030, Toray TGP-H-060, Toray TGP-H-090, Toray TGP-H-120, Freudenberg H23C6, Freudenberg H15C13, Freudenberg H15C14, Freudenberg H14C10, Freudenberg H14CX483, Freudenberg H14CX653, Freudenberg H23C2, Freudenberg H23CX653, Freudenberg H24CX483, Freudenberg H23C6,Freudenberg H23C8, Freudenberg H24C5, Freudenberg H23C3, Avcarb MB-30, Avcarb GDS5130, Avcarb GDS2130, Avcarb GDS3250, Avcarb GDS3260, Avcarb GDS2230, Avcarb GDS2240, Avcarb GDS2255, Avcarb GDS2185, AvCar 1071, AvCarb 1698, AvCarbon1209, AvCarb 1185, AvCarb 1186, AvCarb 7497, AvCarb T1819, AvCarb T1820, AvCarb T1824, AvCarbon 1071, AvCarb 1698, AvCarb 1209, AvCarb 1185, AvCarb 1186, AvCarb 1186, AvCarb T1819, AvCarb T1820, AvCarb T1824, AvCarb EP40, AvCarb P75, AvCarb EP55, AvCarbon EP4OT, AvCarb P75T, AvCarb EP55T, AvCarb MGL190, AvCarb MGL280, AvCarbMGL370. The metal-based porous support can be selected from titanium, stainless steel, Ni, Cu or any other suitable metal and can be under the form of mesh, frit, foam or plate of any thickness or porosity.

In specific embodiments of the invention, the electrolyzer can include a separating element to separate specific generated chemicals from others. The separating element can be one or more traps on the cathodic and/or anodic outputs of the electrolyzer which separates liquid outputs from gaseous outputs. It can also be more complex systems known by those skilled in the art for the purpose of efficient product separation. The separating element can be a separating area between the anode area and the cathode area configured to separate the volume of generated chemicals from the electrolyzer. The separating area can be a separating layer. Efficient physical separation of the anode area and cathode area may allow easier separation of the gases released from each section of the reactor. The separator can be an ion-conducting polymeric separator, a non-ion conducting polymeric separators, a non-ionically charged polymer, a non-ionically charged separator, an ionomer solution coated onto the electrodes, a diaphragm, a ceramic-containing material, a non-charged separator scaffold, a mixed ceramic-organic compound separator, or any other separator. Separation may occur through the use of ion-exchange membranes, which favor the diffusion of either anions (in an anion-exchange membrane) or cations (in a cation-exchange membrane), or a bipolar membrane (including a mixture of cation- and anion-exchange membranes), or other types of separators, such as diaphragms, ceramic-containing materials (in particular mixed ceramic/organic compounds), or non-charged separator scaffolds. Anion-exchange membrane can comprise an organic polymer with positively charged functionality, such as, but not limited to, imidazolium, pyridinium, or tertiary amines. This allows facile migration of negatively charged hydroxide ions (OH⁻) produced during carbon monoxide reduction from the cathode to the anode. The use of this layer also prevents the crossover of other gases from the cathode to the separating layer. Cation-exchange membranes can comprise an organic polymer with negatively charged functionality such as, but not limited to, sulfonate groups. Diaphragms or non-charged separators can be materials derived from insulating materials which may be charged with an ion-conducting electrolyte to facilitate charge transfer between electrodes. Ceramic-containing materials may be a purely ceramic or mixed polymer and ceramic material. Ceramic-polymer mixes can reach higher temperatures than purely organic polymers and may take advantage of ion-exchange functionality in the polymer to pass charge between electrodes. The thickness of the membranes can be chosen precisely to control the transport rates of species such as anions, cations, and neutral species such as alcohols and water during operation.

In specific embodiments of the invention, the system can include an electrolyte that will facilitate the transportation of ions and provide ions that promote the reactions. In particular, the electrolyte may be a concentrated alkaline solution such as a solution of hydroxide-containing salt such as, but not limited to, potassium, sodium or cesium hydroxide with concentrations such as (0.01 molarity (M), 0.05 M, 0.1 M, 0.2 M, 0.5 M, 1 M, 2 M, 3 M, 4 M, 5 M, 6 M, 7 M, 8 M, 9 M and 10 M). The use of concentrated alkaline solution brings down the energy requirement of the overall reaction. Alkali metal cations (such as Li, Na, K, Cs, Rb) may be used as counter-cations. This electrolyte may contain oxidation substrates other than water or hydroxide, such as dihydrogen, alcohols, glycerol, other organic materials, and other oxidizable feedstocks.

In specific embodiments of this invention, a separation system is used to separate the liquid products. In particular, there is a distillation-based method used to separate acetate evolved as an acetate-containing solution or acetic acid-containing solution depending on the electrolyte pH. Such separation unit can use heat recovered from the electric arc furnace off-gas.

In specific embodiments of the invention, the flow field can comprise a ladder, single or multiple serpentines, interdigitated patterns, pillars, bio-inspired leaf-like shapes or a mixture thereof. An electrolysis cell can also include polar plates as further discussed in this disclosure. The performance of the electrolyzer can be modulated by altering the characteristics of the flow field, specifically to prevent the buildup of condensed phases that slow down the mass transport of carbon monoxide and the efflux of liquid products. For example, a larger number of flow field channels in the same area can be used to extract liquid products more efficiently from the cathode, relative to a flow field with a lower density of channels.

In specific embodiments of the invention, the electrolyzer can be operated at elevated temperature and pressure to promote the stability and performance of the electrolyzer by improving carbon monoxide mass transport and product efflux. Elevated temperature can serve to evaporate liquid products present in the cathode catalyst layer, while elevated pressure can mitigate the intrusion and retention of liquids in the cathode catalyst layer. The electrolyzer can be operated under elevated pressure at both the anode and cathode compartments, or only in one compartment to precisely manage liquid and gas crossover in the electrolyzer. The heat for the elevated temperature can be harvested from the electric arc furnace such as by maintaining a temperature of the off-gas at a certain level while it is being processed and delivered to the electrolyzer in a carbon monoxide rich form.

Carbon monoxide humidification upstream of the carbon monoxide electrolyzer can be controlled in specific embodiments of the invention. Several humidification process can be applied to a carbon monoxide stream, but not limited to, (1) steam injection in a gas stream, (2) membrane water/gas contact module, (3) water gas bubbler, (4) other water/gas contact systems including, but not limited to, sprayers, and packed column. For solution (2), (3), (4), the gas outlet water content will mainly depend on the system operating conditions (pressure and temperature), the contact time and the exchange area between the two phases. In that case, to increase water content in the gas stream, it can be necessary to heat the inlet gas stream and/or the water put into contact with the gas. Solution (1) includes a steam generation module which can use, as primary energy, electricity or fuel gas in a boiler or heat harvested from the electric arc furnace. The generated steam is then mixed with the gas stream to control relative humidity. Gas stream can be pre-heated to avoid condensation in the mixing area.

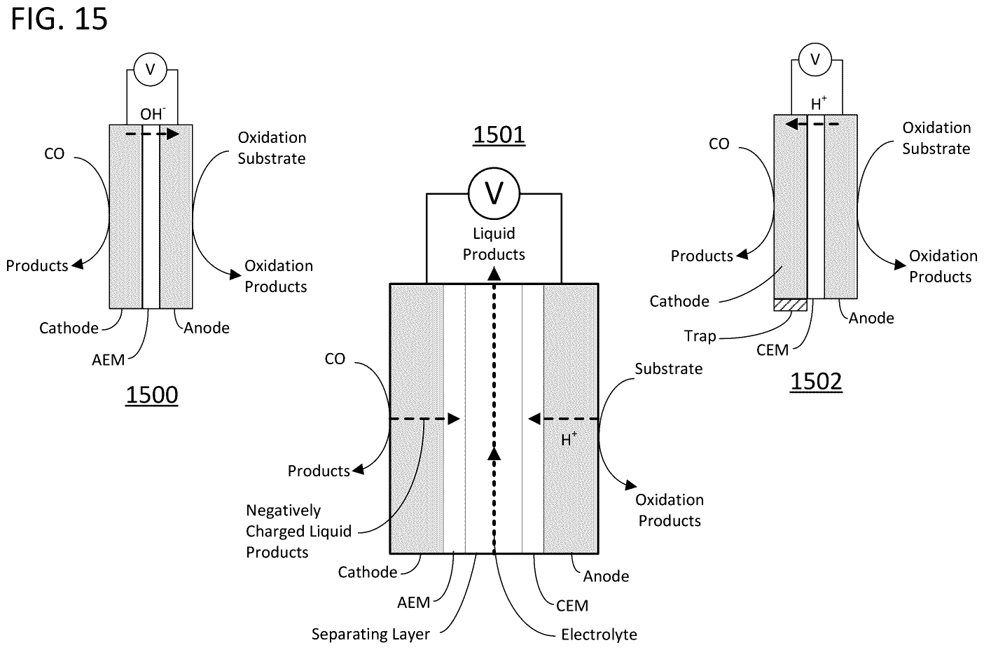

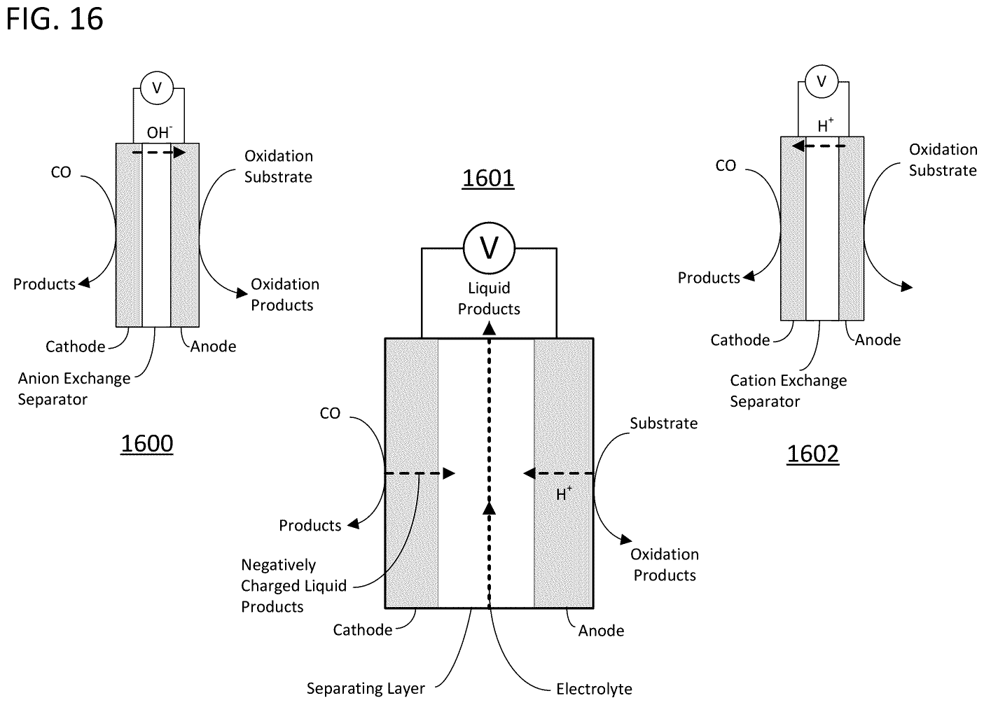

FIGS. 15 and 16 illustrate examples of reactions that can be conducted in accordance with the electrolyzer assemblies described herein. In the diagrams, only single cells are represented for clarity, but these could easily be assembled in a plurality of cells such as in a stack. In the diagrams, a carbon monoxide electrolyzer comprises a cathode comprising a gas-diffusion layer and a copper-based catalyst, and the anode comprises a nickel material of any shape (such as, but not limited to, a foam, a mesh, a deposit onto a conductive porous transport layer (PTL), etc.). In this case, the carbon monoxide reduction products include one or more of the following: ethylene (C₂H₄), ethanol (C₂H₅OH), acetic acid (CH₃COOH), propylene (C₃H₆), propanol (C₃H₈O), oxalic acid (COOH—COOH), acrylic acid (C₂H₃COOH), glyoxylic acid (COH—COOH) produced according to the following carbon monoxide reduction reactions:

In neutral/alkaline conditions:

2CO+6H₂O+8e⁻ à CH₂CH₂+8OH⁻ (9)

2CO+7H₂O+8e⁻ à CH₃CH₂OH+8OH⁻ (10)

2CO+4H₂O+4e⁻ à CH₃COOH+4OH⁻ (11)

3CO+5H₂O+6e⁻ à C₂H₃COOH+6OH⁻ (12)

3CO+9H₂O+12e−à C₃H₆+12OH⁻ (13)

3CO+10H₂O+12e−à C₃H₈O+12OH⁻ (14)

In acidic conditions:

2CO+8H⁺+8e⁻ à CH₂CH₂+2H₂O (15)

2CO+8H⁺+8e⁻ à CH₃CH₂OH+H₂O (16)

2CO+4H⁺+4e⁻ à CH₃COOH (17)

3CO+6H⁺+6e⁻ à C₂H₃COOH+H₂O (18)

3CO+12H⁺+12e⁻ à C₃H₆+3H₂O (19)

3CO+12H⁺+12e⁻à C₃H₈O+2H₂O (20)

In specific embodiments, the carbon monoxide stream is mixed with other gas or liquid compounds to generate higher added value products at the cathode. In one such embodiment, imines, amines, nitrogen oxides or ammonia are added to react with carbon monoxide, or an intermediate formed during its reduction, to form amide bonds or N-rich organic compounds, such as amino acids or urea. Examples of such reactions are:

2CO+3H2O+NH3+4e−→CH3CONH2+4OH⁻ in neutral/alkaline conditions (21)

2CO+4H⁺+NH₃+4e⁻ à CH₃CONH₂+H₂O in acidic conditions (22)

In specific embodiments, the oxidation reaction at the anode is selected from the group consisting of reactions undertaken in an acidic environment and reactions undertaken in an alkaline environment such as, but not limited to, anodic reactions in an acidic environment such as:

2H₂O→O₂+4H⁺+4e⁻ (23)

H₂→2H⁺+2e⁻ (24)

Cl⁻→Cl₂+2e⁻ (25)

Br⁻→Br₂+2e⁻ (26)

I⁻→I₂+2e⁻ (27)

C₃H₈O₃(glycerol)→C₃H₆O₃(glyceraldehyde)+2H⁺+2e⁻ (24)

C₃H₈O₃(glycerol)+H₂O→C₃H₅O₄⁻(glycerate)+5H⁺+4e⁻ (25)

C₃H₈O₃(glycerol)+ 3/2H₂O→ 3/2C₂H₃O₃⁻+ 13/2H⁺+5e⁻ (26)

C₃H₈O₃(glycerol)+3H₂O→3HCOO⁻(formate)+11H⁺+8e⁻ (27)

C₃H₈O₃(glycerol)+3H₂O→ 3/2C₂O₄²⁻+14H⁺+11e⁻ (28)

and anodic reactions in neutral/alkaline environments such as:

4OH⁻→O₂+2H₂O+4e⁻ (29)

H₂+2OH⁻→2H₂O+2e⁻ (30)

Cl⁻→Cl₂+2e⁻ (31)

Br⁻→Br₂+2e⁻ (32)

I⁻→I₂+2e⁻ (32)

C₃H₈O₃(glycerol)+2OH⁻→C₃H₆O₃(glyceraldehyde)+2H₂O+2e⁻ (34)

C₃H₈O₃(glycerol)+5OH⁻→C₃H₅O₄⁻(glycerate)+4H₂O+4e⁻ (35)

C₃H₈O₃(glycerol)+ 13/2OH⁻→ 3/2C₂H₃O₃⁻+5H₂O+5e⁻ (36)

C₃H₈O₃(glycerol)+11OH⁻→3HCOO⁻(formate)+8H₂O+8e⁻ (37)

C₃H₈O₃(glycerol)+14OH⁻→ 3/2C₂O₄²⁻+11H₂O+11e⁻ (38)

C₂H₅OH+5OH⁻→CH₃COO⁻+4H₂O+4e⁻ (39)

C₃H₇OH+5OH⁻→CH₃CH₂COO⁻+4H₂O+4e⁻ (40)

In specific embodiments of the inventions disclosed herein, the carbon monoxide electrolyzer includes one or more ion exchange membranes chosen among anion-exchange membranes (such as, but not limited to, commercial Ionomr®, Orion®, Sustainion®, Piperion®, and ionomer anion-exchange membranes), proton-exchange membranes (such as but not limited to Nation®, Aquivion®, or commercial membranes), bipolar membranes (such as, but not limited to, Fumasep® FBM and Xion®). In specific embodiments of the invention, the membrane in an anion-exchange membrane is prepared using N-bearing monomers. In the example of reactor 1500, the electrolyzer includes an anion exchange membrane and hydroxide moves from the cathode to the anode. The oxidation product depends on the oxidation substrate, while the product harvested from the cathode output can be any of the generated chemicals mentioned above. In the example of reactor 1502, the electrolyzer includes a cation exchange membrane and protons move from the anode to the cathode. The oxidation product again depends on the oxidation substrate, while the product harvested from the cathode output can be any of the generated chemicals mentioned above.

In specific embodiments of the inventions disclosed herein, the electrolyzer can include a separating layer. In the example of reactor 1501, the carbon monoxide electrolyzer comprises a central separating layer in which an electrolyte fluid is circulated allowing the collection of liquid carbon-monoxide-reduction products that migrate from the cathode toward the central separating layer. In specific embodiments, the central separating layer is either separated from the cathode by an anion-exchange membrane or from the anode by a cation-exchange membrane, or both membranes are present. In the example of reactor 1501, both membranes are present. In this example, useful products can be harvested both from the liquid stream in from the separating layer and a gaseous stream from the cathode output. For example, the carbon monoxide could be used by the electrolyzer to produce one or more of the following: ethylene (C₂H₄), ethanol (C₂H₅OH), acetic acid (CH₃COOH), propylene (C₃H₆), propanol (C₃H₈O). In a specific embodiment, the main targeted product is ethylene (in the gaseous product stream). In another specific embodiment, the main targeted products are ethylene (in the gaseous product stream) and ethanol (in the liquid product stream). In another specific embodiment, the main targeted products are ethylene (in the gaseous product stream) and acetic acid/acetate (in the liquid product stream). For example, in reactor 1502, a trap is located at the cathodic output which separates liquid products from gaseous products such that they can both be collected. In these examples, the oxidation occurring at the anode could be water/hydroxide oxidation, dihydrogen oxidation, or chloride oxidation. Notably, in a physical system the trap is located on the connection to the outlet of the cathode such as to piping that is connected to the cathode, and the trap is drawn connected to the cathode area for diagrammatic purposes only.

The examples illustrated in FIG. 16 are similar to those of FIG. 15 in terms of the overall theory of the reactor. However, the approaches in FIG. 16 operate without the use of exchange membranes and instead operate with separating layers that achieve similar effects. Reactor 1600 is similar to that of reactor 1500 in that hydroxide ions move from the cathode to the anode and generated products can be harvested from the cathode output. Reactor 1601 is similar to that of reactor 1501 in that the separating layer includes a liquid electrolyte and useful products can be harvested both from the output of the separating layer in liquid form and from an output of the cathode area in fluid form. Reactor 1602 is similar to reactor 1502 in that protons migrate across the separating layer and useful products can be harvested from the output of the cathode.

In specific embodiments of the invention, a porous diaphragm can be used in the electrolyzer as a separation element to achieve separation. The diaphragm can be saturated with an electrolyte which allows ions to cross between the cathode and anode. The diaphragm can allow ions to cross from the anode to the cathode and/or ions to cross from the cathode to the anode.

In specific embodiments of the invention, a carbon monoxide stream is mixed with at least one other chemical such as other gas or liquid compounds to generate higher added value products at the cathode of an electrolyzer. The carbon monoxide stream can be mixed with such additive chemicals at the time the carbon monoxide is supplied to the electrolyzer. In one such embodiment, imines, amines, nitrogen oxides, or ammonia are added to react with carbon monoxide, or an intermediate formed during its reduction, to form amide bonds or nitrogen rich organic compounds, such as amino acids. In another embodiment, aromatic or aliphatic acids/aldehydes/alcohols are added to react with the carbon monoxide, or an intermediate formed during its reduction, to form hydrocarbons, alcohols or organic acids. In another embodiment, aromatic or aliphatic olefins or hydrocarbons are added to react with the carbon monoxide, or an intermediate formed during its reduction, to form hydrocarbons, alcohols or organic acids. These reactions can be combined with any of the reactors mentioned above. For example, the oxidation occurring at the anode can be water oxidation, hydroxide oxidation, dihydrogen oxidation, or halide oxidation.

In specific embodiments, the carbon-monoxide-depleted off-gas stream obtained after the separation from the carbon-monoxide-rich stream to be fed to the carbon monoxide electrolyzer, is further purified to obtain a carbon dioxide stream. Such carbon dioxide stream can be fed to a reverse water gas shift (RWGS) reactor that takes both hydrogen and such purified carbon dioxide stream to produce a stream containing carbon monoxide, unreacted carbon dioxide and dihydrogen and some methane. The carbon monoxide obtained from the RWGS reactor can then be further purified and fed to the carbon monoxide electrolyzer in addition to the carbon-monoxide-rich stream obtained directly from the off-gas purification. The tandem reactor architectures disclosed herein can be designed to operate continuously with carbon monoxide being produced by the RWGS reactor in time to supply the carbon monoxide electrolyzer with a sufficient supply of carbon monoxide (e.g., when the electric arc furnace is not producing an off-gas because it is not operating in a phase that does so). The control system for the tandem reactor could include safeguards to shut down the reactor, or portions thereof, upon detecting the presence or absences of certain chemicals in the reactor.

In specific embodiments, the carbon monoxide electrolyzer evolves dihydrogen at the cathode as a parasitic reaction that competes with the targeted reduction of carbon monoxide to the valuable carbon-based chemicals. In that case, the evolved dihydrogen can be separated from the stream of valuable chemicals to be either valorized as is or fed back to the RGWS reactor to minimize the requirements in hydrogen for the conversion of the electric arc furnace off-gas derived or captured carbon dioxide.

In specific embodiments of the inventions disclosed herein, one or more separators can be located on the fluid connections between the reactor components. The separators can be designed to separate out specific chemicals from a fluid stream in the fluid connection. For example, a volume of carbon monoxide can be separated from a volume of carbon dioxide at the output of the electric arc furnace off-gas uptakes or at an output of a RWGS reactor using at least one separator unit. Then at least one separator in this example can be an acid scrubber and the electrolyzer can be an alkaline reactor. In the case of the separator from the electric arc furnace off-gas, unreacted oxygen must be removed through a specific separation to minimize the amount of oxygen fed to the carbon monoxide electrolyzer as the reduction of oxygen is favored compared to carbon monoxide reduction and leads to wasteful consumption of energy for the unwanted conversion of oxygen into water.

In specific embodiments of the invention, the carbon monoxide can be separated from trace chemicals left over in the output of the electric arc furnace off-gas uptake or of the RWGS reactor. The carbon monoxide can be separated using various approaches such as separating with membranes, cryogenic separating, separating methods based on variant physical or chemical properties of the components of the output of the RWGS reactor or the electric arc furnace off-gas, separation based on pressure-swing adsorption, temperature-swing adsorption, vacuum- or vacuum-pressure swing adsorption, or separation based on absorption.

In the case where a RWGS is present, a separating system can, for example, be used on the output of the RWGS reactor to first cool the output to remove impurities and then heat the output fluid to allow purified dihydrogen to evaporate through a membrane that filters out carbon monoxide. Any carbon dioxide or dihydrogen filtered out of the output of the RWGS reactor can be fed back to serve a feedstock to the RWGS reactor. Any carbon monoxide filtered out of the output of the electrolyzer can be fed back to serve as a feedstock to the RWGS reactor. Dihydrogen filtered out of the output of the electrolyzer can be fed back to serve as a feedstock to the RWGS reactor.

The carbon monoxide concentrated exhaust gas from an electric arc furnace can reach a relatively high temperature (500-1500° C.), contains particulate matter (PM) as fly ashes, carbon particles, and dust, and contains gas contaminants as SOx or NOx and other gas components as hydrogen, water, nitrogen, oxygen and carbon dioxide.

Several process units can be set on the off-gas stream to make the gas compatible with the specification of a carbon monoxide electrolyzer. First the gas may be cooled and made free of PM (5 mg/Nm3). For that purpose, a cyclone unit can be installed directly on the hot stream. Gas can enter the cyclone through a tangential inlet at high velocity (10-30 m/s) and swirl in the cylindrical section of the vessel at the top following by the conical section at the bottom. Then, a spiral vortex can be created making the heavier solid particle fall and the gas stream flow up. Cyclones are usually designed for a cut diameter (d50, d90) meaning that respectively 50% or 90% of the particles with a higher diameter are removed. This cut diameter is usually comprised between 10 and 50 μm meaning that the unit won't remove all the particulate matter. Downstream units can be used to achieve less than 5 mg/Nm3.

Different options are available to cool the gas. Shell and tube heat exchangers using thermal oil or hot water as coolant can be used. This kind of heat exchanger can be divided into different radiative and convective sections using different coolants or not. The temperature gradient between hot gas and coolant must be controlled. A particle recovery section, comprising of an endless screw, can be set between the radiative and the convective sections. The outlet temperature of the gas can be around 250° C.-300° C. to remain above the water dew point temperature and avoid condensation.

Downstream of the cooling unit, a bag filter can be installed to remove remaining particles. PTFE sleeves can be installed for that purpose. Their maximum operating condition is around 250° C. which corresponds to their degradation temperature. The particles contained in the gas stream will stop and accumulate on the sleeves creating a filter cake. Once the pressure drop through this filter is too high, pressurized air or inert gas is introduced at counter current to make the cake fall. The solid is then removed from the vessel during operation. A coating material, introduced upstream of the bag filter in the form of powder, can be used to capture/remove a part of the gas impurities especially sulfur species, HCl, HCN, and phosphine through the filtration cake layer. The coating material can be, but is not limited to, activated carbon, calcium hydroxide, or lime. In embodiments in which the electric arc furnace is being used to produce calcium carbide, the lime or calcium hydroxide can be advantageously sourced from the calcium carbide process. Ceramic or metallic cartridges can be used for the same purpose than PTFE sleeves. The main advantage is that they can be operated at higher temperature, but their price can be significantly higher.

A water quench unit (e.g., jet venturi scrubber) can also be set downstream, upstream or be used instead of the bag filtration unit. In a water quench unit, liquid water is mixed with hot gas. Heat is consumed by the vaporization of the injected water, and the heat is exchanged with cold water to lower the gas temperature. This kind of unit allows cooling the gas to low temperature (<80° C.) while removing the water condensate and particulate matter (PM) and a part of the gas species soluble in water (e.g., HCl, HCN).

Gas fans or gas blowers can be installed upstream, downstream or in the middle of the filtering and cooling units to ensure gas flowing. All the units set downstream of the electric arc furnace and upstream of the gas fan can be operated under atmospheric pressure, and all the units downstream of the gas fan can be operated above atmospheric pressure. Operating units under atmospheric pressure limits the risk of carbon monoxide leaks in the process environment. However, air (oxygen) can be accidentally introduced in the process pipe creating an explosive mix. Operating under atmospheric pressure or above can be done according to the risk evaluation and the cost difference between a simple gas fan and a gas fan able to be operated at high temperature in the presence of dust.

Downstream of the filtration and cooling units, a carbon monoxide concentrated stream can still contain some gas impurities such as, but not limited to, sulfur compounds (SOx), light hydrocarbons (e.g., Benzene), phosphine (PH3), nitrogen oxides (NOx), or carbon dioxide and other main components such as hydrogen.

A fixed bed filled with dedicated sorbent can be set to remove different kind of impurities, especially sulfur compounds as SOx, COS and H2S, and phosphine. Most commonly, activated carbon impregnated with specific species as, but not limited to, potassium iodide, potassium permanganate, and metal oxides (such as, but not limited to, CuO, FeO, MgO) are used to treat sulfur compounds. The impregnation of these specific species promotes sulfur oxidation into elemental sulfur, which accumulates on the sorbent surface. Adsorption capacities can be as high as 80-100 w/w %. Once the filter is saturated, the sorbent must be replaced. Phosphine can also be removed by being impregnated with activated carbon, HCl, KNO₃, Cu species, or hexanediol for example. This chemical species allows for the catalytic oxidation of PH₃ except for the copper which reacts with phosphine to form Cu₃P₂. Molecular sieves (zeolite) or alumina impregnated with different species (e.g., CaCl₂) can also adsorb PH₃. This sorbent must be selective through PH₃ over carbon monoxide to be used. Oxygen presence in the exhaust gas even in small quantity (<1% v) will significantly increase the adsorption capacity for sulfur products and phosphine. Non-impregnated activated carbons can be used to remove oxygenated volatile organic compounds (OVCs) and inorganic compounds. Their adsorption capacity is quite low compared to impregnated activated carbon, but they can be regenerated by flowing the vessel at counter current with steam or warm inert gas. Heat recovered in the heat exchangers on the exhaust gas can be valorized for that purpose. Other processes such as chemical absorption can also be implemented to remove sulfur compounds.

After the removal of inorganic impurities and OVCs, the carbon monoxide concentrated stream can contain hydrogen (2-40% v dry basis), carbon dioxide (0-5% v dry basis), and nitrogen (1-30% v dry basis). The stream also contains water according to the temperature and the pressure of the stream.

Carbon dioxide is an issue for the carbon monoxide electrolyzer operation and must be removed prior to that unit. The reaction of carbon dioxide with the anolyte (e.g., OH⁻) produces carbonate and bicarbonate species which lead to the formation of salt precipitates or regions of high salt concentration that foul electrolyzer components and lead to substantially degraded performance. For the cathode section, these precipitates are generally hygroscopic and impede efficient gas and liquid transport across the cathode. This is exacerbated by the fact that the mass diffusivity of carbon dioxide in water is approximately four orders of magnitude lower than its mass diffusivity in the gas phase. Salts formed from the reaction of alkaline media with carbon dioxide gases can also precipitate within the membrane pores and impede efficient ion transport.

Commercially available carbon dioxide removal units can be based on cryogenic processes, membrane separation modules, swing adsorption processes or absorption processes.

Hydrogen and nitrogen are inert gases when fed to carbon monoxide electrolyzers. Their presence only impacts the partial pressure of carbon monoxide at the cathode side. Accordingly, these compounds can be removed upstream or downstream of the carbon monoxide electrolyzer according to their inlet concentration and the nature and the purity of produced species. Other amounts of dihydrogen may be produced in the carbon monoxide electrolyzer. In such cases, it could be more efficient to separate the total amount of dihydrogen coming from the electric arc furnace and electrolyzer downstream of the electrolyzer.

The separating steps and processes described above can take on various forms. The separation system may conduct one or more of multiple separation/purification steps including any technology available for the targeted purification/separation. The separation system can include separation units based on but not limited to membrane technologies including but not limited to dense polymeric membranes, ultrafiltration and nano-filtration membranes, facilitated-transport membranes, metallic membranes, hollow fiber pervaporation membranes, cryogenic technologies, adsorption technologies including but not limited to physisorption and chemisorption technologies, absorption technologies, including physical absorption technologies and chemical absorption technologies, with operation techniques such as, but not limited to, vacuum pressure swing, temperature swing, pressure swing, arid pressure swing, coupled pressure and temperature swing, and electric swing. Chemical adsorbents that can be used include but are not limited to amine-based adsorbents (amine grafted or impregnated solids), metal oxides, metal salts, double salts, and hydrotalcites. Physical adsorbents that can be used include but are not limited to materials such as carbon-based materials, mesoporous silica, zeolites, zeolitic imidazolate frameworks (ZIF's), metalorganic frameworks (MOF's), blended adsorbents.

The removal of carbon dioxide gases from a gas mixture using the techniques detailed above will need to bring carbon dioxide concentrations below a certain desired threshold, depending on process conditions. This acid gas removal process may require a combination of techniques, or multiple stages of separator units to more thoroughly scrub acid gases from the gas mixture. For example, to reduce carbon dioxide concentrations down to less than 1% in a carbon monoxide-rich gas stream leaving a carbon monoxide-producing process, one or more pressure swing adsorption subunits, absorption subunits, or membrane separation subunits may be combined in series or parallel to ensure a high degree of acid gas removal, depending on process conditions required.

Chemical absorption technologies to remove acid gas (e.g., carbon dioxide) can include those that use methods relying on reversible complexation with a soluble metal complex, or alkaline and/or amine-bearing solutions that use the chemical action of base equivalents to capture acid gases. Most commonly, chemical and physical absorption process units separating carbon dioxide from a gas stream are composed by two main equipment, (1) the absorption tower and (2) the regeneration tower. In the absorption tower, the inlet gas is fed at the bottom while the liquid is fed at the top at counter current. The column internals include, but are not limited to, structured packing, random packing, trays, gas and liquid distributors, liquid sprayers which aim to maximize the exchange area between the liquid and the gas phases. Absorption columns can be operated at 10-80° C. and at 1-80 bars according to the used solvent. The loaded solvent exits the absorption tower by the bottom, is then pumped and pre-heated before entering the regeneration tower. A regeneration tower can be a stripping column or distillation column which includes a reboiler and a condenser. The absorbed carbon dioxide can be released at the column top by the effect of the temperature increase and/or of the pressure decrease. The regeneration tower can be operated at 80-150° C. and 1-10 bars. Lean solvent can be pumped and cooled before entering the absorption column and completing the loop. Chemical based solvents can be, but not limited to methylethanolamine (MEA), dimethylethanolamine (DEA), methyl diethanol amine (MDEA), piperazine (PZ), soda (NaOH), KOH, a solvent blend. Physical solvent can be dimethyl ether (DME), methanol, a solvent blend.

Swing adsorption techniques are used to physically or chemically adsorb a species in a fluid line to separate it from other gases. Swing adsorption is generally non-oxidative to the carbon monoxide and dihydrogen present in the gas stream. Such techniques use an adsorbent selective for one or more of the molecules in a fluid line and achieve separation through the following steps: the first is the adsorption of the one species, while all other species pass through the adsorbent, and the second is a regeneration, wherein an increase in temperature or/and a decrease in pressure is used to extract the adsorbed species from the adsorbent material. Several swing adsorption separators, usually between two and ten, may be operated in parallel to allow continuous separation to occur and to minimize the specific power consumption. The adsorbent material can operate via a chemical or physical mechanism. Chemical adsorbents that can be used include, but are not limited to, amine-based adsorbents (amine grafted or impregnated solids), metal oxides, metal salts, double salts and hydrotalcites. Physical adsorbents that can be used include but are not limited to materials such as activated carbons, carbon molecular sieves, mesoporous silica, zeolites, zeolitic imidazolate frameworks (ZIF's), metal organic frameworks (MOF's), or blended adsorbents. Swing adsorption processes can be applied to, but not limited to, carbon dioxide removal, oxygen removal, carbon oxide/dihydrogen separation, nitrogen removal, volatile organic chemical removal, methane/carbon oxide separation, gas drying, and a mix of the previous application according to the sorbent material nature, number of different sorbent layers and the operating conditions.

Membrane separation uses an extended surface comprising a polymeric species for the movement/restriction of a particular species in a fluid line. Membrane separation is generally non-oxidative to the carbon monoxide and dihydrogen present in the gas stream. The separator may comprise several layers of the membrane surface to achieve effective separation. At commercial scale, membrane can be arranged, but not limited to, in a hollow fiber module, in a spiral wound module. The separation is achieved through a favorable chemical interaction of the membrane with the substance to be removed from the fluid line or through a size of pore tailored for the exclusion of larger molecules within the fluid. The different gas species either end on the permeate side, meaning they have gone through the membrane layers leading to pressure drops, or in the retentate side. The separation driving force can be the pressure gradient or/and the concentration gradient between the permeate and the retentate side. These processes may require several independent stages of compressor and membrane units to achieve full purification of the fluid line and to reach the largest recovery rate of the desired species. Membranes can be applied to, but not limited to, carbon dioxide removal, oxygen removal, nitrogen removal, dihydrogen/carbon monoxide separation, olefin removal, gas drying, and a mix of the previous application according to the membrane material, the number of membrane stages and the operating conditions.

The separation unit used to separate the carbon monoxide contained in the electric arc furnace off-gas can be, but not limited to, an absorption unit using liquid solvent (e.g., CO-SORB process reactor) or a PSA (e.g., CO-PSA) using specific sorbent material. The separating stream containing carbon dioxide, oxygen, nitrogen, dihydrogen and other gases present in the electric arc furnace off-gas can be vented and/or valorized for their calorific content. The purified carbon monoxide is then sent to an electrolyzer to produce any valuable product, among other, ethylene, ethanol or acetic acid. The carbon dioxide can be captured in the separation unit and then sent to a different system for further processing. In specific embodiments, extra heat from the electric arc furnace can be used to produce steam for amine regeneration or to regenerate solid sorbent in a temperature swing absorption separator.

The carbon monoxide gas mixture to be purified and fed into to the carbon monoxide electrolyzer, depending on the production process, can be water saturated at the stream pressure and temperature or relative humidity can be as high as 80%-100% at the considered pressure and temperature. To avoid water condensation in pipes, gas compressors, process units, water can be fully or partially removed until a defined temperature dew point. Pipes and process units can be insulated or heat traced (electrically or through sealed envelope). Several processes can be used to remove water such as, but not limited to: (1) a heat exchanger using cool refrigerant to condense water; (2) a physical absorption unit using physical solvents such as, but not limited to, methanol, glycol (Mono ethylene glycol (MEG), diethylene glycol (DEG), triethylene glycol (TEG), tetraethylene glycol (TREG)); (3) a membrane based processes which are selective for water removal, (4) an adsorption filter using sorbent such as, but not limited to, activated alumina, zeolite (3A, 4A), silica gel. Solution (1) cannot reduce the gas water dew point below 0-5° C. Solution (2), (3) and (4) can reduce the gas water dew point between −10° C. and −50° C. meaning less than 10 ppmv of water.

Carbon monoxide rich gas can be compressed, upstream or downstream the separation units, prior to introduction into the electrolyzer. Compressor technologies can be, but not limited to, centrifugal or volumetric. Volumetric technologies include, but not limited to, membrane compressor, screw compressor and reciprocating compressor. The technology choice will depend on the gas flowrate and on the required outlet pressure. Knowing that the maximum compression ratio through a compressor is commonly taken at 3, between 1 and 5 compression stages may be needed to reach the required pressure. Inter-stage cooling steps may then be necessary.

While some systems include two separation systems and a single electrolyzer, many different variations are possible. For example, a single, or multiple separating systems can be connected in series to separate out chemicals such as carbon dioxide from reaching the input of the electrolyzer. The separating can be conducted to ever increasing levels of purity and one or more of the multiple separating systems can be coupled to a single electrolyzer for the delivery of carbon monoxide. As another example, multiple such separating systems can be coupled with a set of electrolyzers that are configured to accept cathodic inputs with different levels of carbon monoxide volume or concentration.

In specific embodiments, the electric arc furnace can use direct reduced iron (DRI) produced from a DRI furnace. The DRI furnace can use the DRI iron to produce steel. The DRI furnace can be fueled with different reductant sources and in particular gases such as methane or dihydrogen. The dihydrogen can be recovered from the off-gas of the electric arc furnace or from the operation of the electrolyzers disclosed herein. When methane is used, great amounts of carbon monoxide are released in the exhaust gases which can be valorized through a carbon monoxide electrolyzer towards high-valuable chemicals. The DRI off-gas can also contain some dihydrogen that can be used as an oxidation substrate for any electrochemical process. Alternatively, the dihydrogen can be used as a feedstock of a RWGS process, in addition to carbon dioxide, to produce carbon monoxide fed to the electrolyzer. In specific embodiments, the DRI off-gas also contains carbon dioxide in addition to the dihydrogen and at least part of each is provided to the RWGS reactor to produce carbon monoxide which is then fed to the carbon monoxide electrolyzer. The carbon monoxide can also be sourced from a basic oxygen furnace or blast furnace from the more traditional route to make steel.

While the specification has been described in detail with respect to specific embodiments of the invention, it will be appreciated that those skilled in the art, upon attaining an understanding of the foregoing, may readily conceive of alterations to, variations of, and equivalents to these embodiments. For example, while the example of an electric arc furnace was used in this disclosure, other furnaces can be used in place of or along with the electric arc furnace in the embodiments disclosed herein. The furnaces can be one or more furnaces selected from a group consisting of: a direct reduced iron furnace, a blast oxygen furnace, and an electric arc furnace. The disclosure of volumes of chemicals in this disclosure is not meant to refer to a physically isolated volume as it is possible for a volume of dihydrogen to exist with a volume of carbon dioxide in a single physical volume in the form of a volume of syngas. Although examples in the disclosure were generally applied to industrial chemical processes, the same approaches are applicable to chemical processing of any scale and scope. These and other modifications and variations to the present invention may be practiced by those skilled in the art, without departing from the scope of the present invention, which is more particularly set forth in the appended claims.

1. A method comprising:
operating an electric arc furnace;
generating, via operation of the electric arc furnace, a volume of carbon monoxide;
separating, using a non-oxidative separating process, the volume of carbon monoxide from a volume of carbon dioxide in an off-gas from the electric arc furnace;
recovering heat from the off-gas and cooling the off-gas using a heat recovery unit and a non-oxidative cooling process;
capturing a second volume of carbon dioxide;
regenerating a media employed for the capture of the second volume of carbon dioxide using the heat recovered from the heat recovery unit;
supplying the volume of carbon monoxide to a cathode area of a carbon monoxide electrolyzer to be used as a reduction substrate; and
generating, using the carbon monoxide electrolyzer, the reduction substrate, and an oxidation substrate, a volume of generated chemicals;
wherein: (i) the off-gas is cooled prior to supplying the volume of carbon monoxide to the cathode area of the carbon monoxide electrolyzer; and (ii) the volume of generated chemicals is at least one of: a volume of hydrocarbons, a volume of organic acids, a volume of alcohol, a volume of olefins and a volume of N-rich organic compounds.

2. The method of claim 1, wherein:
the carbon monoxide electrolyzer is a low temperature electrolyzer.

3. (canceled)

4. The method of claim 1, further comprising:
storing the volume of carbon monoxide in a gas storage downstream of the electric arc furnace and upstream of the carbon monoxide electrolyzer.

5. The method of claim 1, further comprising:
decreasing a rate of supply for the volume of carbon monoxide to the carbon monoxide electrolyzer when an arc of the electric arc furnace is off; and
increasing the rate of supply for the volume of carbon monoxide to the carbon monoxide electrolyzer when the arc is on.

6. The method of claim 1, wherein:
the electric arc furnace is operated intermittently;
a rate of supply for the volume of carbon monoxide to the carbon monoxide electrolyzer is variable according to a rate control; and
the carbon monoxide electrolyzer has a startup time of less than ten minutes.

7. The method of claim 1, wherein:
the volume of carbon monoxide is generated in the off-gas from the electric arc furnace;
the off-gas includes a volume of dihydrogen; and
the volume of dihydrogen is provided to at least one of: (i) an anode area of the carbon monoxide electrolyzer to be used as the oxidation substrate; and (ii) a direct reduction furnace, wherein direct reduced iron from the direct reduction furnace is used during the operating of the electric arc furnace.

8. The method of claim 1, further comprising:
generating a volume of dihydrogen; and
producing direct reduced iron using a direct reduction furnace and the dihydrogen;
wherein the direct reduced iron is used during the operating of the electric arc furnace and the volume of dihydrogen is generated by a parasitic reaction in the cathode area of the carbon monoxide electrolyzer.

9. The method of claim 1, wherein:
the generating of the volume of generated chemicals also generates a volume of oxygen gas; and
the oxidation substrate is one of: water; metal hydroxide; and hydroxide.

10. The method of claim 9, further comprising:
supplying the volume of oxygen gas to the electric arc furnace by injecting the volume of oxygen into a molten bath in the electric arc furnace; and
forming a slag on the molten bath using the volume of oxygen.

11. The method of claim 9, further comprising:
supplying the volume of oxygen gas to at least one heater of the electric arc furnace; and
heating the electric arc furnace using the volume of oxygen gas and the at least one heater.

12. The method of claim 11, further comprising:
heating the volume of oxygen gas using the off-gas from the electric arc furnace, wherein the volume of carbon monoxide is generated in the off-gas; and at least one of: (i) supplying the volume of oxygen gas to the electric arc furnace by injecting the volume of oxygen gas into a molten bath in the electric arc furnace; and (ii) supplying the volume of oxygen gas to at least one heater of the electric arc furnace.

13. The method of claim 1,
wherein the non-oxidative separating process uses heat recovered from the electric arc furnace.

14. (canceled)

15. (canceled)

16. The method of claim 1, further comprising:
supplying the volume of carbon dioxide to a reverse water gas shift reactor to produce a second volume of carbon monoxide; and
supplying the second volume of carbon monoxide to the cathode area of the carbon monoxide electrolyzer to be used as the reduction substrate.

17. The method of claim 16, wherein:
the reverse water gas shift reactor uses heat recovered from the electric arc furnace.

18. The method of claim 1, further comprising:
calcinating limestone in a lime kiln to produce lime and carbon dioxide;
producing calcium carbide in the electric arc furnace using the lime;
supplying the volume of carbon dioxide to a reverse water gas shift reactor to produce a second volume of carbon monoxide; and
supplying the second volume of carbon monoxide to the cathode area of the carbon monoxide electrolyzer to be used as the reduction substrate.

19. The method of claim 1, further comprising:
generating acetylene from calcium carbide in a hydrolysis reaction, wherein the operating of the electric arc furnace produces the calcium carbide in the electric arc furnace; and
separating, using a separator, the volume of carbon dioxide from the volume of carbon monoxide in the off-gas from the electric arc furnace;
wherein the separator uses heat from the hydrolysis reaction.

20. The method of claim 1, further comprising:
generating acetylene from a volume of calcium carbide in a hydrolysis reaction, wherein the operating of the electric arc furnace produces the volume of calcium carbide in the electric arc furnace, and wherein the hydrolysis reaction produces a volume of calcium hydroxide; and
scrubbing, using the calcium hydroxide, the volume of carbon dioxide from the volume of carbon monoxide in the off-gas from the electric arc furnace.

21. The method of claim 1, further comprising:
producing calcium carbide in the electric arc furnace.

22. The method of claim 1, wherein:
the operating of the electric arc furnace produces a molten metal in the electric arc furnace.

23. The method of claim 22, further comprising:
operating a direct reduced iron furnace via combustion of a hydrocarbon to generate direct reduced iron;
wherein: (i) the direct reduced iron is used by the electric arc furnace to produce the molten metal; (ii) the combustion of the hydrocarbon generates a second volume of carbon monoxide; and (iii) the second volume of carbon monoxide is supplied to the cathode area of the carbon monoxide electrolyzer to be used as the reduction substrate.

24. The method of claim 1, further comprising:
mixing the volume of carbon monoxide with a volume of at least one additive chemical;
wherein the volume of carbon monoxide has been mixed with the volume of at least one additive chemical when the volume of carbon monoxide is supplied to the carbon monoxide electrolyzer.

25. The method of claim 24, wherein:
the volume of additive chemical is one of an imine, an amine, a nitrogen oxide and ammonia; and
the volume of generated chemicals is a volume of amino acids.

26. A system comprising:
an electric arc furnace;
an off-gas port of the electric arc furnace for an off-gas including a volume of carbon monoxide;
a separator configured to separate, using a non-oxidative separating process, the volume of carbon monoxide from a volume of carbon dioxide in the off-gas;
a heat recovery unit configured to recover heat from the off-gas and cool the off-gas using a non-oxidative cooling process;
a carbon dioxide capture system configured to capture a second volume of carbon dioxide;
a media employed by the carbon dioxide capture system for the capture of the second volume of carbon dioxide, wherein the system is configured to regenerate the media using the heat recovered from the heat recovery unit;
a carbon monoxide electrolyzer having an anode area and a cathode area; and
at least one fluid connection;
wherein: i) the volume of carbon monoxide is routed from the separator to the cathode area to be used as a reduction substrate using the at least one fluid connection; (ii) the carbon monoxide electrolyzer is configured to generate, using the reduction substrate and an oxidation substrate, a volume of generate chemicals; (iii) the off-gas is cooled prior to supplying the volume of carbon monoxide to the cathode area of the carbon monoxide electrolyzer; and (iv) the volume of generated chemicals is at least one of: a volume of hydrocarbons, a volume of organic acids, a volume of alcohol, a volume of olefins and a volume of N-rich organic compounds.

27. The system of claim 26, further comprising:
a gas storage; and
a set of valves;
wherein: (i) the gas storage is part of the at least one fluid connection; and (ii) the set of valves is configured to allow the volume of carbon monoxide to flow from the off-gas port to the gas storage in a first configuration and to the volume of carbon monoxide to flow from the gas storage to the cathode area in a second configuration.

28. The system of claim 26, further comprising:
a control system, wherein the control system is configured to:
decrease a rate of supply for the volume of carbon monoxide to the carbon monoxide electrolyzer when an arc of the electric arc furnace is off; and
increase the rate of supply for the volume of carbon monoxide to the carbon monoxide electrolyzer when the arc is on.

29. (canceled)

30. (canceled)
